(19) United States
(12) Patent Application Publication
(10) Pub. No.: US 2024/0209182 A1
(43) Pub. Date: Jun. 27, 2024

(54) HARDENER, ADHESIVE COMPOSITION, ADHESIVE FILM FOR CIRCUIT CONNECTION, CONNECTED STRUCTURE, AND METHOD FOR PRODUCING CONNECTED STRUCTURE

(71) Applicants: Resonac Corporation, Tokyo (JP); Sika Technology AG, Baar (CH)

(72) Inventors: Toshimitsu MORIYA, Tokyo (JP); Takayuki ICHIMURA, Tokyo (JP); Masayuki MIYAJI, Tokyo (JP); Ryohta KOBAYASHI, Tokyo (JP); Kazunori ISHIKAWA, Kanagawa (JP); Tsubasa ITO, Kanagawa (JP)

(21) Appl. No.: 18/555,190

(22) PCT Filed: Apr. 14, 2022
(86) PCT No.: PCT/JP2022/017841

§ 371 Date: Oct. 12, 2023

(30) Foreign Application Priority Data
Apr. 16, 2021 (JP) 2021-069569
Aug. 6, 2021 (JP) 2021-130105

(51) Int. Cl. C08K 5/3432; C09J 7/30; C09J 9/02; C09J 11/04; C09J 11/06; C09J 163/04

(52) U.S. Cl. CPC C08K 5/3432; C09J 7/30; C09J 9/02; C09J 11/04; C09J 11/06; C09J 163/04

(57) ABSTRACT

A curing agent containing a pyridinium salt, in which the pyridinium salt has a benzyl group at a 1-position and has an electron-withdrawing group at a 2-position, and the benzyl group has an electron-donating group. An adhesive composition containing a pyridinium salt and a cationic polymerizable compound, in which the pyridinium salt has a benzyl group at a 1-position and has an electron-withdrawing group at a 2-position, and the benzyl group has an electron-donating group. An adhesive film for circuit connection including an adhesive layer formed from the adhesive composition.

### TECHNICAL FIELD

The present disclosure relates to a curing agent, an adhesive composition, an adhesive film for circuit connection (an anisotropic conductive adhesive film or a conductive adhesive film), a connected structure, and a method for producing a connected structure.

### BACKGROUND ART

As a circuit connection material for electrically connecting electrodes in a pressurizing direction to each other by heating and pressurizing facing circuits, an adhesive film for circuit connection in which conductive particles are dispersed in an epoxy-based adhesive or an acrylic adhesive is known. The adhesive film for circuit connection is used, for example, in electrical connection between a TCP (Tape Carrier Package) or COF (Chip On Flex) on which a semiconductor driving a liquid crystal display (LCD) is mounted and an LCD panel or between a TCP or COF and a printed circuit board.

In recent years, in order to increase manufacturing efficiency, an adhesive that can be cured at a low temperature and in a short time has been examined. For example, Patent Literature 1 describes an adhesive composition that can be cured at a relatively low temperature (for example, 150° C. to 170° C.) and in a short time (for example, within 10 seconds).

### CITATION LIST

### Patent Literature

Patent Literature 1: Japanese Unexamined Patent Publication No. 2017-214472

### SUMMARY OF INVENTION

### Technical Problem

In recent years, from the viewpoint of weight reduction and designability of a display, the frame of the display is narrowed, and for example, the demand for products such as smartphones, in which the frame of the display hardly exists is increasing. Furthermore, a place on which an electronic component such as a driver IC or TCP for driving a display is mounted is also narrowed, and particularly in the display whose frame is narrow, the electronic component may be mounted in the vicinity of a display unit. When the heat resistance of a member of the display unit is low, troubles may occur in the member of the display unit due to heat generated when the electronic component is mounted. Therefore, an adhesive composition that can be cured at a low temperature is required. Furthermore, from the viewpoint of increasing manufacturing efficiency, it is required to cure the adhesive composition at a lower temperature.

Therefore, an object of the present disclosure is to provide a curing agent capable of curing an adhesive composition at a lower temperature (for example, 120° C.). Furthermore, another object of the present disclosure is to provide an adhesive composition, an adhesive film for circuit connection, a connected structure, and a method for producing a connected structure, which use the curing agent.

### Solution to Problem

An aspect of the present disclosure is a curing agent containing a pyridinium salt, in which the pyridinium salt has a benzyl group at a 1-position and has an electron-withdrawing group at a 2-position, and the benzyl group has an electron-donating group.

According to the curing agent which is the aspect of the present disclosure, an adhesive composition can be cured at a lower temperature (for example, 120° C.).

In the curing agent, the electron-withdrawing group may be a cyano group or a halogeno group.

In the curing agent, the electron-donating group may be an alkyl group or an alkoxy group.

In the curing agent, the number of electron-donating groups of the benzyl group may be 3, and the electron-donating group may be an alkyl group.

In the curing agent, the pyridinium salt may contain a pyridinium cation and an anion, and the anion may be B(C₆F₅)₄⁻.

Another aspect of the present disclosure is an adhesive composition containing a pyridinium salt and a cationic polymerizable compound, in which the pyridinium salt has a benzyl group at a 1-position and has an electron-withdrawing group at a 2-position, and the benzyl group has an electron-donating group. According to the adhesive composition which is the aspect of the present disclosure, the adhesive composition can be cured at a lower temperature (for example, 120° C.).

In the adhesive composition, the electron-withdrawing group may be a cyano group or a halogeno group.

In the adhesive composition, the electron-donating group may be an alkyl group or an alkoxy group.

In the adhesive composition, the number of electron-donating groups of the benzyl group may be 3, and the electron-donating group may be an alkyl group.

In the adhesive composition, the pyridinium salt may contain a pyridinium cation and an anion, and the anion may be B(C₆F₅)₄⁻.

The cationic polymerizable compound may contain an epoxy compound.

A content of the curing agent may be 0.1 to 40 parts by mass based on 100 parts by mass of the cationic polymerizable compound.

The adhesive composition may further contain conductive particles.

Still another aspect of the present disclosure is an adhesive film for circuit connection, including an adhesive layer formed from the above-described adhesive composition. According to the adhesive film for circuit connection which is the aspect of the present disclosure, the adhesive composition can be cured at a lower temperature (for example, 120° C.).

A content of the curing agent may be 1 to 20% by mass based on a total mass of the adhesive film for circuit connection.

The adhesive film for circuit connection may include a first adhesive layer and a second adhesive layer laminated on the first adhesive layer, and at least one of the first adhesive layer and the second adhesive layer may be a layer formed from the above-described adhesive composition. That is, the adhesive film for circuit connection may include a first adhesive layer and a second adhesive layer laminated on the first adhesive layer, and at least one of the first adhesive layer and the second adhesive layer may contain the above-described curing agent and the above-described cationic polymerizable compound.

Still another aspect of the present disclosure is a connected structure including a first circuit member having a first electrode, a second circuit member having a second electrode, and a connection portion disposed between the first circuit member and the second circuit member and electrically connecting the first electrode and the second electrode to each other, in which the connection portion contains a cured product of the above-described adhesive film for circuit connection.

Still another aspect of the present disclosure is a method for producing a connected structure, the method including a step of interposing the above-described adhesive film for circuit connection between a first circuit member having a first electrode and a second circuit member having a second electrode, and thermocompression bonding the first circuit member and the second circuit member to electrically connect the first electrode and the second electrode to each other.

### Advantageous Effects of Invention

According to the present disclosure, it is possible to provide a curing agent capable of curing an adhesive composition at a lower temperature (for example, 120° C.). Furthermore, according to the present disclosure, it is possible to provide an adhesive composition, an adhesive film for circuit connection, a connected structure, and a method for producing a connected structure, which use the curing agent.

### BRIEF DESCRIPTION OF DRAWINGS

FIG. 1 is a schematic cross-sectional view illustrating an embodiment of an adhesive film for circuit connection.

FIG. 2 is a schematic cross-sectional view illustrating an embodiment of an adhesive film for circuit connection.

FIG. 3 is a schematic cross-sectional view illustrating an embodiment of a connected structure.

FIG. 4 is a schematic cross-sectional view illustrating a method for producing the connected structure of FIG. 3.

FIG. 5 shows DSC measurement results of an adhesive film for circuit connection of Example 1.

FIG. 6 shows DSC measurement results of an adhesive film for circuit connection of Example 2.

FIG. 7 shows DSC measurement results of an adhesive film for circuit connection of Example 3.

FIG. 8 shows DSC measurement results of an adhesive film for circuit connection of Example 4.

FIG. 9 shows DSC measurement results of an adhesive film for circuit connection of Example 5.

FIG. 10 shows DSC measurement results of an adhesive film for circuit connection of Example 6.

FIG. 11 shows DSC measurement results of an adhesive film for circuit connection of Example 7.

FIG. 12 shows DSC measurement results of an adhesive film for circuit connection of Example 8.

FIG. 13 shows DSC measurement results of an adhesive film for circuit connection of Example 9.

FIG. 14 shows DSC measurement results of an adhesive film for circuit connection of Comparative Example 1.

FIG. 15 shows DSC measurement results of an adhesive film for circuit connection of Comparative Example 2.

FIG. 16 shows DSC measurement results of an adhesive film for circuit connection of Comparative Example 3.

FIG. 17 shows DSC measurement results of an adhesive film for circuit connection of Comparative Example 5.

FIG. 18 shows DSC measurement results of an adhesive film for circuit connection of Comparative Example 6.

FIG. 19 shows DSC measurement results of an adhesive film for circuit connection of Comparative Example 7.

### DESCRIPTION OF EMBODIMENTS

Hereinafter, embodiments of the present disclosure will be described in detail. Note that, the present disclosure is not limited to the following embodiments.

In a numerical range described in the present specification, an upper limit value or a lower limit value of the numerical range may be replaced with a value shown in Examples. Furthermore, the lower limit value and the upper limit value of the numerical range are each optionally combined with a lower limit value or an upper limit value of another numerical range. In the notation of the numerical range “A to B”, the numerical values A and B at both ends are included in the numerical range as the lower limit value and the upper limit value, respectively. In the present specification, for example, the description “10 or more” means “10” and “numerical values more than 10”, and the same applies to the case of different numerical values. Furthermore, for example, the description “10 or less” means “10” and “numerical values less than 10”, and the same applies to the case of different numerical values. Furthermore, each of components and materials exemplified in the present specification may be used singly or may be used in combination of two or more kinds thereof, unless otherwise specified. In the present specification, when a plurality of substances corresponding to each component exist in the composition, the content of each component in the composition means the total amount of the plurality of substances that exist in the composition, unless otherwise specified. Furthermore, in the present specification, the term “(meth)acrylate” means at least one of acrylate and methacrylate corresponding thereto.

### <Pyridinium Salt>

An embodiment of the present disclosure is a pyridinium salt having a benzyl group at a 1-position and having an electron-withdrawing group at a 2-position in which the benzyl group has an electron-donating group. That is, the pyridinium salt has a pyridine ring and a benzene ring and has an electron-withdrawing group disposed at the ortho-position with respect to the nitrogen atom of the pyridine ring, and the benzene ring has an electron-donating group. The pyridinium salt having a benzyl group at a 1-position and having an electron-withdrawing group at a 2-position in which the benzyl group has an electron-donating group (hereinafter, also referred to as “pyridinium salt A”) may be a compound configured by a pyridinium cation and an anion. Note that, in the present specification, the 1-position of the pyridinium salt or pyridinium cation means the position of the nitrogen atom in the pyridine ring of the pyridinium salt or pyridinium cation.

### <Curing Agent>

The pyridinium salt A can be used, for example, as a curing agent. That is, another embodiment of the present disclosure is a curing agent containing a pyridinium salt, in which the pyridinium salt has a benzyl group at a 1-position and has an electron-withdrawing group at a 2-position, and the benzyl group has an electron-donating group. When the curing agent contains the pyridinium salt A, an adhesive composition can be cured at a lower temperature (for example, 120° C.).

The pyridinium salt A may be, for example, a compound represented by General Formula (1) below.

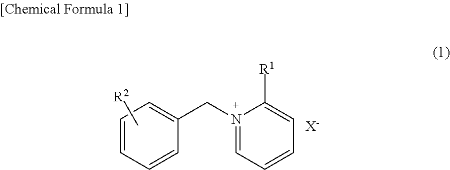

[In Formula (1), R¹ represents an electron-withdrawing group, R² represents an electron-donating group, and X⁻ represents an anion.]

Examples of the electron-withdrawing group at the 2-position of the pyridinium salt A include a cyano group, a halogeno group, a nitro group, a carbonyl group, a carboxy group, and a sulfo group. Examples of the halogeno group include a fluoro group, a chloro group, a bromo group, and an iodo group. The electron-withdrawing group may be a cyano group or a halogeno group and may be a cyano group or a chloro group, from the viewpoint that the activity of the curing agent is increased so that the adhesive composition can be cured in a shorter time. The pyridinium salt A may contain an electron-withdrawing group other than the potential-withdrawing group disposed at the 2-position. The number of electron-withdrawing groups of the pyridinium salt A may be 3 or less, 2 or less, or 1.

Examples of the electron-donating group of the benzyl group disposed at the 1-position of the pyridinium salt A include an alkyl group, an alkoxy group, a hydroxyl group, an amino group, and an alkylamino group. Examples of the alkyl group include a methyl group, an ethyl group, a n-propyl group, and an isopropyl group. Examples of the alkoxy group include a methoxy group and an ethoxy group. The electron-withdrawing group may be an alkyl group or an alkoxy group and may be a methyl group or a methoxy group, from the viewpoint that the activity of the curing agent is increased so that the adhesive composition can be cured in a shorter time. The benzene ring may contain a plurality of electron-donating groups, and the number of electron-donating groups of the benzyl group disposed at the 1-position of the pyridinium salt A may be 1 or more, 2 or more, or 3 or more, and may be 3. The benzyl group disposed at the 1-position of the pyridinium salt A may have at least one electron-donating group at a 4-position (the 4-position when the binding position of the benzyl group with the pyridine ring is the 1-position; the para-position with respect to the binding position of the benzyl group with the pyridine ring).

When the number of electron-donating groups of the benzyl group disposed at the 1-position of the pyridinium salt A is 3, all of three electron-donating groups may be an alkyl group and may be a methyl group. That is, still another embodiment of the present disclosure is a pyridinium salt having a benzyl group at a 1-position and having an electron-withdrawing group at a 2-position, in which the benzyl group has three electron-donating groups, all of which are an alkyl group. The pyridinium salt A may have an alkyl group as an electron-donating group at each of the 2-position, the 4-position, and the 6-position of the benzyl group when the binding position of the benzyl group with the pyridine ring is the 1-position. Since the curing agent contains a pyridinium salt in which the number of electron-donating groups of the benzyl group disposed at the 1-position of the pyridinium salt A is 3 and all of the electron-donating groups are an alkyl group (or a methyl group), an adhesive film using such a curing agent has excellent physical properties (for example, elastic modulus). Therefore, the adhesive film using such a curing agent can achieve, for example, both of excellent adhesion to a circuit member and excellent peelability of a substrate from the adhesive film. Furthermore, the adhesive film using such a curing agent is, for example, excellent in storage stability, and even in a case where the adhesive film is stored for a certain period of time (for example, 15 hours at 40° C.), excellent adhesion to a circuit member and excellent peelability of a substrate from the adhesive film are likely to be maintained. The reason for this is considered that, when the number of electron-donating groups of the benzyl group disposed at the 1-position of the pyridinium salt A is 3, the adhesive film has a well-balanced structure which prevents deterioration during storage for a certain period of time (for example, 15 hours at 40° C.) (is excellent in storage stability) while maintaining low-temperature curability.

Examples of the pyridinium cation of the pyridinium salt A include 2-cyano-1-(4-methoxybenzyl)pyridinium cation, 2-chloro-1-(4-methoxybenzyl)pyridinium cation, 2-bromo-1-(4-methoxybenzyl)pyridinium cation, 2-cyano-1-(4-methylbenzyl)pyridinium cation, 2-chloro-1-(4-methylbenzyl)pyridinium cation, 2-bromo-1-(4-methylbenzyl)pyridinium cation, 2-cyano-1-(2,4,6-trimethylbenzyl)pyridinium cation, 2-chloro-1-(2,4,6-trimethylbenzyl)pyridinium cation, and 2-bromo-1-(2,4,6-trimethylbenzyl)pyridinium cation. The pyridinium cation of the pyridinium salt A may be at least one selected from the group consisting of 2-cyano-1-(4-methoxybenzyl)pyridinium cation, 2-chloro-1-(4-methoxybenzyl)pyridinium cation, 2-cyano-1-(2,4,6-trimethylbenzyl)pyridinium cation, and 2-chloro-1-(2,4,6-trimethylbenzyl)pyridinium cation, from the viewpoint that the adhesive composition can be cured in a shorter time.

Examples of the anion of the pyridinium salt A include SbF₆⁻, PF₆⁻, PF_{X}(CF₃)_{6-X}⁻ (provided that, X is an integer of 1 to 5), BF₄⁻, B(C₆F₅)₄⁻, RSO₃⁻ (provided that, R is an alkyl group having 1 to 3 carbon atoms or a substituted or unsubstituted aryl group), C(SO₂CF₃)₃⁻, N(SO₂CF₃)₂⁻, O(SO₂CF₃)⁻, and B(C₆H₃(CF₃)₂)₄⁻ (provided that, the CF₃ group is substituted at the 3- and 5-position of the phenyl group). The anion of the pyridinium salt A may be B(C₆F₅)₄⁻ from the viewpoint of having excellent connection resistance even after a high-temperature and high-humidity test (for example, 85° C., 85% RH, 250 hours). That is, still another embodiment of the present disclosure is a pyridinium salt configured by a pyridinium cation having a benzyl group at a 1-position and having an electron-withdrawing group at a 2-position, and an anion, in which the anion is B(C₆F₅)₄⁻.

The pyridinium salt A may be a compound obtained by combining the above-described pyridinium cation and the above-described anion. That is, the pyridinium salt A may contain at least any of the pyridinium cations described above and any of the anions described above. The pyridinium salt A may be at least one selected from the group consisting of 2-cyano-1-(4-methoxybenzyl)pyridinium tetrakis(pentafluorophenyl)borate, 2-chloro-1-(4-methoxybenzyl)pyridinium tetrakis(pentafluorophenyl)borate, 2-cyano-1-(2,4,6-trimethylbenzyl)pyridinium tetrakis(pentafluorophenyl)borate, and 2-chloro-1-(2,4,6-trimethylbenzyl)pyridinium tetrakis(pentafluorophenyl)borate, from the viewpoint that the adhesive composition can be cured in a shorter time.

The content of the pyridinium salt A in the curing agent may be 80% by mass or more, 90% by mass or more, or 95% by mass or more, based on the total mass of the curing agent, and may be 100% by mass (an embodiment in which the curing agent is substantially composed of the pyridinium salt A).

The curing agent may contain a pyridinium salt other than the pyridinium salt A. The content of the pyridinium salt other than the pyridinium salt A in the curing agent may be 20% by mass or less, 10% by mass or less, or 5% by mass or less, based on the total mass of the curing agent, and may be 0% by mass (an embodiment in which the curing agent is substantially composed of the pyridinium salt A).

The curing agent containing the pyridinium salt A can be obtained, for example, by a production method including a step of reacting at least one of a pyridine compound having an electron-withdrawing group at the 2-position, a benzyl chloride compound having an electron-donating group, and a benzyl bromide compound having an electron-donating group, and an iodide salt of an alkali metal (for example, sodium iodide) in a solvent (for example, acetonitrile) to obtain pyridinium iodide having a pyridine ring and benzene ring and a step of reacting the obtained pyridinium iodide and an anion salt in a solvent (for example, dichloromethane) to obtain a pyridinium salt A.

The pyridine compound having an electron-withdrawing group at the 2-position may be the above-described pyridine compound having an electron-withdrawing group at the 2-position, and examples thereof include 2-cyanopyridine and 2-chloropyridine.

The benzyl chloride compound having an electron-donating group may be the above-described benzyl chloride compound having an electron-donating group, and examples thereof include 4-methoxybenzyl chloride and 2,4,6-trimethylbenzyl chloride. The benzyl bromide compound having an electron-donating group may be the above-described benzyl bromide compound having an electron-donating group, and examples thereof include 4-methoxybenzyl bromide and 2,4,6-trimethylbenzyl bromide.

The anion salt may be a compound capable of introducing the anion of the pyridinium salt A, and may be, for example, a lithium salt, sodium salt, potassium salt, or cesium salt of the anion of the pyridinium salt A described above.

In the step of obtaining pyridinium iodide, the reaction may be performed, for example, at room temperature (20° C. to 30° C.). The reaction time may be, for example, 10 to 50 hours or 20 to 30 hours. After completion of the reaction, the used solvent may be removed by washing the obtained pyridinium iodide with acetone, distilled water, or the like and vacuum-drying the pyridinium iodide.

In the step of obtaining pyridinium iodide, the yield of the pyridinium iodide may be 40% or more, 55% or more, 70% or more, or 80% or more. The yield of the pyridinium iodide refers to a ratio of an actually obtained amount with respect to a maximum amount of pyridinium iodide that can be obtained from a raw material used to synthesize pyridinium iodide.

In the step of obtaining the pyridinium salt A, the reaction may be performed, for example, at room temperature (20° C. to 30° C.). The reaction time may be, for example, 1 to 15 hours or 1 to 5 hours. After completion of the reaction, the used solvent may be removed by washing the obtained pyridinium salt A with acetone, distilled water, or the like and vacuum-drying the pyridinium salt A.

In the step of obtaining the pyridinium salt A, the yield of the pyridinium salt A may be 70% or more, 80% or more, or 85% or more. The yield of the pyridinium salt A refers to a ratio of an actually obtained amount with respect to a maximum amount of the pyridinium salt A that can be obtained from pyridinium iodide used to synthesize the pyridinium salt A.

Whether the pyridinium salt A is obtained can be checked by measuring the obtained compound by nuclear magnetic resonance spectrum (¹H-NMR). Specifically, whether the pyridinium salt A is obtained can be checked by the method described in Examples below.

### <Adhesive Composition>

Still another embodiment of the present disclosure is an adhesive composition containing a pyridinium salt A and a cationic polymerizable compound. Furthermore, still another embodiment of the present disclosure is an adhesive composition containing a curing agent containing a pyridinium salt A and a cationic polymerizable compound.

The cationic polymerizable compound may be, for example, a compound which reacts with the pyridinium salt A (or the curing agent containing the pyridinium salt A) by heating to be cross-linked. Examples of the cationic polymerizable compound include an epoxy compound, a vinyl ether compound, and an oxetane compound. The cationic polymerizable compound may contain an epoxy compound. The cationic polymerizable compound may be used singly or may be used in combination of two or more kinds thereof.

Examples of the epoxy compound include a bisphenol A-type epoxy resin, a bisphenol S-type epoxy resin, a bisphenol F-type epoxy resin, a phenol novolac-type epoxy resin, a cresol novolac-type epoxy resin, a bisphenol A novolac-type epoxy resin, a bisphenol F novolac-type epoxy resin, a tetramethyl bisphenol A-type epoxy resin, 3′,4′-epoxycyclohexylmethyl-3,4-epoxycyclohexane carboxylate (bi-7-oxabicyclo[4,1,0]heptane), 3,4-epoxycyclohexylmethyl (meth)acrylate, (3,3′,4,4′-diepoxy)bicyclohexyl, dicyclopentadiene dimethanol diglycidyl ether, xylene-novolac-type glycidyl ether, and a biphenyl-type epoxy resin. The epoxy compound may contain at least one selected from the group consisting of a bisphenol A-type epoxy resin, a tetramethyl bisphenol A-type epoxy resin, dicyclopentadiene dimethanol diglycidyl ether, xylene-novolac-type glycidyl ether, and an alicyclic epoxy resin. The epoxy compound may contain a glycidyl ether-based compound. From the viewpoint of further improving low-temperature curability, the epoxy compound may contain an alicyclic epoxy resin. Furthermore, from the viewpoint that both of low-temperature curability and favorable storage stability can be easily achieved, the epoxy compound may not contain an alicyclic epoxy resin.

The oxetane compound can be used without particular limitation as long as it is a compound having one or more oxetane ring structures in the molecule. From the viewpoint of further improving low-temperature curability, the cationic polymerizable compound may contain an oxetane compound. Furthermore, from the viewpoint that both of low-temperature curability and favorable storage stability can be easily achieved, the cationic polymerizable compound may not contain an oxetane compound. The cationic polymerizable compound may contain an epoxy compound and an oxetane compound. Furthermore, from the viewpoint that both of low-temperature curability and favorable storage stability can be easily achieved, the cationic polymerizable compound may contain only one of an epoxy compound and an oxetane compound. Examples of a case where the cationic polymerizable compound contains only one of an epoxy compound and an oxetane compound include a case where one selected from an epoxy compound and an oxetane compound is used singly as a cationic polymerizable compound and a case where one selected from an epoxy compound and an oxetane compound and a cationic polymerizable compound such as a vinyl ether compound are used in combination.

The content of the cationic polymerizable compound may be 10% by mass or more, 30% by mass or more, 40% by mass or more, or 50% by mass or more, based on the total mass of the adhesive composition, from the viewpoint of securing the curability of the adhesive composition. The content of the cationic polymerizable compound may be 70% by mass or less, 65% by mass or less, or 50% by mass or less, based on the total mass of the adhesive composition, from the viewpoint of securing the formability of the adhesive composition. From these viewpoints, the content of the cationic polymerizable compound may be 10 to 70% by mass based on the total mass of the adhesive composition.

The content of the curing agent containing the pyridinium salt A in the adhesive composition may be 1% by mass or more, 2% by mass or more, 3% by mass or more, 4% by mass or more, 4.5% by mass or more, 5% by mass or more, 6% by mass or more, 7% by mass or more, 8% by mass or more, 10% by mass or more, 11% by mass or more, or 11.5% by mass or more, based on the total mass of the adhesive composition, from the viewpoint of sufficiently promoting a curing reaction. The content of the curing agent containing the pyridinium salt A in the adhesive composition may be 20% by mass or less, 15% by mass or less, 12% by mass or less, 10% by mass or less, 9% by mass or less, 8% by mass or less, 7% by mass or less, 6% by mass or less, 5% by mass or less, or 4% by mass or less, based on the total mass of the adhesive composition, from the viewpoint of improving the physical properties of a cured product. From these viewpoints, the content of the curing agent containing the pyridinium salt A in the adhesive composition may be 1 to 20% by mass or 1 to 15% by mass based on the total mass of the adhesive composition. The content of the pyridinium salt A in the adhesive composition may be within the above content range.

The content of the curing agent containing the pyridinium salt A in the adhesive composition may be 1% by mass or more, 2% by mass or more, 3% by mass or more, 4% by mass or more, 4.5% by mass or more, 5% by mass or more, 6% by mass or more, 8% by mass or more, 9% by mass or more, 10% by mass or more, 11% by mass or more, 11.5% by mass or more, 12% by mass or more, or 13% by mass or more, based on the total mass of the adhesive composition excluding conductive particles, from the viewpoint of sufficiently promoting a curing reaction. The content of the curing agent containing the pyridinium salt A in the adhesive composition may be 30% by mass or less, 25% by mass or less, 20% by mass or less, 15% by mass or less, 14% by mass or less, 12% by mass or less, 10% by mass or less, 9% by mass or less, 8% by mass or less, 7% by mass or less, 6% by mass or less, 5% by mass or less, 4% by mass or less, or 3% by mass or less, based on the total mass of the adhesive composition excluding conductive particles, from the viewpoint of improving the physical properties of a cured product. From these viewpoints, the content of the curing agent containing the pyridinium salt A in the adhesive composition may be 1 to 30% by mass based on the total mass of the adhesive composition excluding conductive particles. The content of the pyridinium salt A in the adhesive composition may be within the above content range.

The content of the curing agent containing the pyridinium salt A in the adhesive composition may be 1% by mass or more, 3% by mass or more, 4% by mass or more, 4.5% by mass or more, 5% by mass or more, 5.5% by mass or more, 6% by mass or more, 7% by mass or more, 8% by mass or more, 10% by mass or more, 11% by mass or more, 11.5% by mass or more, 12% by mass or more, or 14% by mass or more, based on the total mass of the adhesive composition excluding conductive particles and a filler, from the viewpoint of sufficiently promoting a curing reaction. The content of the curing agent containing the pyridinium salt A in the adhesive composition may be 30% by mass or less, 25% by mass or less, 20% by mass or less, 15% by mass or less, 12% by mass or less, 11% by mass or less, 10% by mass or less, 9% by mass or less, 8% by mass or less, 7% by mass or less, 6% by mass or less, 5% by mass or less, 4% by mass or less, or 3% by mass or less, based on the total mass of the adhesive composition excluding conductive particles and a filler, from the viewpoint of improving the physical properties of a cured product. From these viewpoints, the content of the curing agent containing the pyridinium salt A in the adhesive composition may be 1 to 30% by mass based on the total mass of the adhesive composition excluding conductive particles and a filler. The content of the pyridinium salt A in the adhesive composition may be within the above content range.

The content of the curing agent containing the pyridinium salt A in the adhesive composition may be 0.1 parts by mass or more, 0.3 parts by mass or more, 0.5 parts by mass or more, 0.8 parts by mass or more, 1 part by mass or more, 3 parts by mass or more, 5 parts by mass or more, 8 parts by mass or more, 10 parts by mass or more, 12 parts by mass or more, 15 parts by mass or more, 20 parts by mass or more, 24 parts by mass or more, or 28 parts by mass or more, based on 100 parts by mass of the cationic polymerizable compound, from the viewpoint of sufficiently promoting a curing reaction. The content of the curing agent containing the pyridinium salt A may be 40 parts by mass or less, 30 parts by mass or less, 28 parts by mass or less, 25 parts by mass or less, 20 parts by mass or less, 15 parts by mass or less, 12 parts by mass or less, or 10 parts by mass or less, based on 100 parts by mass of the cationic polymerizable compound, from the viewpoint of improving the physical properties of a cured product. From these viewpoints, the content of the curing agent containing the pyridinium salt A may be 0.1 to 40 parts by mass or 1 to 30 parts by mass based on 100 parts by mass of the cationic polymerizable compound. The content of the pyridinium salt A in the adhesive composition may be within the above content range.

The adhesive composition may contain conductive particles. The conductive particles are not particularly limited as long as they are particles having electrical conductivity, and examples thereof include metal particles configured by metals such as gold, silver, palladium, nickel, copper, and solder; conductive carbon particles configured by conductive carbon; and coated conductive particles each containing a core containing non-conductive glass, ceramic, plastic (such as polystyrene), or the like and a coating layer containing the above-described metal or conductive carbon and covering the core. The conductive particles may be coated conductive particles from the viewpoint that the conductive particles are easily deformed by heating and/or pressurizing and electrical conductivity between electrodes can be further improved by increasing a contact area between the electrodes and the conductive particles when the electrodes are electrically connected to each other.

The average particle size of the conductive particles may be 1 μm or more, 2 μm or more, or 2.5 μm or more, from the viewpoint of excellent dispersibility and electrical conductivity. The average particle size of the conductive particles may be 20 μm or less, 15 μm or less, 10 μm or less, 8 μm or less, 6 μm or less, 5.5 μm or less, or 5 μm or less, from the viewpoint of ensuring the insulating property between adjacent electrodes. From these viewpoints, the average particle size of the conductive particles may be 1 to 20 μm, 1 to 15 μm, 1 to 10 μm, 1 to 8 μm, or 1 to 6 μm.

The average particle size of the conductive particles refers to an average value of particle sizes of 300 conductive particles obtained by observing 300 conductive particles contained in the adhesive composition using a scanning electron microscope (SEM) and measuring the particle size of each conductive particle. Note that, in a case where the conductive particles are not spherical, the particle size of the conductive particle is the diameter of a circle circumscribing the conductive particle in an observation image using an SEM.

The particle density of the conductive particles in the adhesive composition may be 100/mm² or more, 1000/mm² or more, or 3000/mm² or more, from the viewpoint of obtaining stable connection resistance. The particle density of the conductive particles in the adhesive composition may be 100000/mm² or less, 50000/mm² or less, or 30000/mm² or less, from the viewpoint of ensuring the insulating property between adjacent electrodes. From these viewpoints, the particle density of the conductive particles in the adhesive composition may be 100 to 100000/mm², 1000 to 50000/mm², or 3000 to 30000/mm².

The content of the conductive particles may be 10% by mass or more, 20% by mass or more, or 25% by mass or more, based on the total mass of the adhesive composition. The content of the conductive particles may be 50% by mass or less, 40% by mass or less, or 30% by mass or less, based on the total mass of the adhesive composition.

The content of the conductive particles may be 10 parts by mass or more, 30 parts by mass or more, 50 parts by mass or more, 70 parts by mass or more, or 90 parts by mass or more, based on 100 parts by mass of the cationic polymerizable compound. The content of the conductive particles may be 200 parts by mass or less, 150 parts by mass or less, 120 parts by mass or less, or 100 parts by mass or less, based on 100 parts by mass of the cationic polymerizable compound.

The adhesive composition may further contain a thermoplastic resin. When the adhesive composition contains the thermoplastic resin, the adhesive composition is easily formed in a film shape. Examples of the thermoplastic resin include a phenoxy resin, a polyester resin, a polyamide resin, a polyurethane resin, a polyester urethane resin, and acrylic rubber. These may be used singly or may be used in combination of two or more kinds thereof.

The weight average molecular weight (Mw) of the thermoplastic resin may be, for example, 5000 or more, 10000 or more, 20000 or more, or 40000 or more, and may be 200000 or less, 100000 or less, 80000 or less, or 60000 or less. The weight average molecular weight of the thermoplastic resin is a value measured by gel permeation chromatography (GPC) and converted using a calibration curve based on standard polystyrene.

The content of the thermoplastic resin may be 5% by mass or more, 15% by mass or more, 20% by mass or more, or 25% by mass or more, based on the total mass of the adhesive composition. The content of the thermoplastic resin may be 40% by mass or less, 30% by mass or less, 20% by mass or less, or 10% by mass or less, based on the total mass of the adhesive composition.

The content of the thermoplastic resin may be 10 parts by mass or more, 30 parts by mass or more, 50 parts by mass or more, or 60 parts by mass or more, based on 100 parts by mass of the cationic polymerizable compound. The content of the thermoplastic resin may be 100 parts by mass or less, 80 parts by mass or less, 60 parts by mass or less, 40 parts by mass or less, or 20 parts by mass or less, based on 100 parts by mass of the cationic polymerizable compound.

The adhesive composition may further contain a coupling agent. When the adhesive composition contains the coupling agent, adhesiveness can be further improved. The coupling agent may be a silane coupling agent, and examples thereof include vinyltrimethoxysilane, vinyltriethoxysilane, 3-glycidoxypropyltrimethoxysilane, 3-glycidoxypropylmethyldiethoxysilane, 3-(meth)acryloxypropylmethyldimethoxysilan, 3-(meth)acryloxypropyltrimethoxysilane, 3-(meth)acryloxypropylmethyldiethoxysilane, 3-(meth)acryloxypropyltriethoxysilane, N-2-(aminoethyl)-3-aminopropylmethyldimethoxysilan, N-phenyl-3-aminopropyltrimethoxysilane, 3-ureidopropyltriethoxysilane, 3-mercaptopropyltrimethoxysilane, 3-isocyanatepropyltriethoxysilane, and condensates of these. These may be used singly or may be used in combination of two or more kinds thereof.

The content of the coupling agent may be 0.5% by mass or more, 1% by mass or more, or 2% by mass or more, based on the total mass of the adhesive composition. The content of the coupling agent may be 15% by mass or less, 10% by mass or less, or 5% by mass or less, based on the total mass of the adhesive composition.

The content of the coupling agent may be 1 part by mass or more, 4 parts by mass or more, or 6 parts by mass or more, based on 100 parts by mass of the cationic polymerizable compound. The content of the coupling agent may be 30 parts by mass or less, 20 parts by mass or less, 10 parts by mass or less, or 6 parts by mass or less, based on 100 parts by mass of the cationic polymerizable compound.

The adhesive composition may further contain a filler. When the adhesive composition contains the filler, connection reliability can be further improved. Examples of the filler include non-conductive fillers (for example, non-conductive particles). The filler may be any of an inorganic filler and an organic filler.

Examples of the inorganic filler include metallic oxide particles such as silica particles, alumina particles, silica-alumina particles, titania particles, and zirconia particles; and metallic nitride particles. These may be used singly or may be used in combination of two or more kinds thereof.

Examples of the organic filler include silicone particles, methacrylate-butadiene-styrene particles, acryl-silicone particles, polyamide particles, and polyimide particles. These may be used singly or may be used in combination of two or more kinds thereof.

The filler may be an inorganic filler and may be silica particles from the viewpoint of improving film moldability and the reliability of the connected structure. The silica particles may be crystalline silica particles or amorphous silica particles, and these silica particles may be synthetic. A method for synthesizing silica may be a dry method or a wet method. The silica particles may contain at least one selected from the group consisting of fumed silica particles and sol-gel silica particles.

The silica particles may be surface-treated silica particles from the viewpoint of excellent dispersibility in the adhesive component. The surface-treated silica particles are obtained, for example, by hydrophobicizing a hydroxyl group on the surface of the silica particles with a silane compound or a silane coupling agent. The surface-treated silica particles may be, for example, silica particles surface-treated with a silane compound such as an alkoxysilane compound, a disilazane compound, or a siloxane compound, and may be silica particles surface-treated with a silane coupling agent.

Examples of the alkoxysilane compound include methyltrimethoxysilane, dimethyldimethoxysilane, phenyltrimethoxysilane, dimethoxyphenylsilane, tetraethoxysilane, methyltriethoxysilane, dimethyldiethoxysilane, phenyltriethoxysilane, n-propyltrimethoxysilane, n-propyltriethoxysilane, hexyltrimethoxysilane, hexyltriethoxysilane, octyltriethoxysilane, decyltrimethoxysilane, 1,6-bis(trimethoxysilyl)hexane, and 3,3,3-trifluoropropyltrimethoxysilane.

Examples of the disilazane compound include 1,1,1,3,3,3-hexamethyldisilazane, 1,3-diphenyltetramethyldisilazane, 1,3-bis(3,3,3,-trifluoropropyl)-1,1,3,3,-tetramethyldisilazane, and 1,3-divinyl-1,1,3,3-tetramethyldisilazane.

Examples of the siloxane compound include tetradecamethylcycloheptasiloxane, decamethylcyclopentasiloxane, hexaphenylcyclosiloxane, octadecamethylcyclononasiloxane, hexadecamethylcyclooctasiloxane, dodecamethylcyclohexasiloxane, octaphenylcyclotetrasiloxane, hexamethylcyclotrisiloxane, heptaphenyldisiloxane, tetradecamethylhexasiloxane, dodecamethylpentasiloxane, hexamethyldisiloxane, decamethyltetrasiloxane, hexamethoxydisiloxane, octamethyltrisiloxane, octamethylcyclotetrasiloxane, 1,3-vinyltetramethyldisiloxane, 2,4,6-trimethyl-2,4,6-trivinylcyclotrisiloxane, 1,3-dimethoxy-1,1,3,3-tetraphenyldisiloxane, 1,1,3,3-tetramethyl-1,3-diphenyldisiloxane, 1,3-dimethyl-1,3-diphenyl-1,3-divinyldisiloxane, 2,4,6,8-tetramethyl-2,4,6,8-tetravinylcyclotetrasiloxane, 1,1,1,3,5,5,5,-heptamethyl-3-(3-glycidoyloxypropyl)trisiloxane, 1,3,5-tris(3,3,3-trifluoropropyl)-1,3,5-trimethylcyclotrisiloxane, 1,1,1,3,5,5,5,-heptamethyl-3-[(trimethylsilyl)oxy]trisiloxane, 1,3,-bis[2-(7-oxabicyclo[4.1.0]heptane-3-yl)ethyl]-1,1,3,3,-tetramethyldisiloxane, 1,1,1,5,5,5-hexamethyl-3-[(trimethylsilyl)oxy]-3-vinyltrisiloxane, 3-[[dimethyl(vinyl)silyl]oxy]-1,1,5,5,-tetramethyl-3-phenyl-1,5-vinyltrisiloxane, octavinyloctasilsesquioxane, and octaphenyloctasilasilsesquioxane.

Examples of the silane coupling agent include vinyltrimethoxysilane, vinyltriethoxysilane, 2-(3,4-epoxycyclohexyl)ethyltrimethoxysilane, 3-glycidoxypropylmethyldimethoxysilane, 3-glycidoxypropyltrimethoxysilane, 3-glycidoxypropylmethyldiethoxysilane, 3-glycidoxypropyltriethoxysilane, p-styryltrimethoxysilane, 3-methacryloxypropylmethyldimethoxysilane, 3-methacryloxypropyltrimethoxysilane, 3-methacryloxypropylmethyldiethoxysilane, 3-methacryloxypropyltriethoxysilane, 3-acryloxypropyltrimethoxysilane, N-2-(aminoethyl)-3-aminopropylmethyldimethoxysilane, N-2-(aminoethyl)-3-aminopropyltrimethoxysilane, 3-aminopropyltrimethoxysilane, 3-aminopropyltriethoxysilane, 3-triethoxysilyl-N-(1,3-dimethyl-butylidene)propylamine, N-phenyl-3-aminopropyltrimethoxysilane, tris-(trimethoxysilylpropyl)isocyanurate, 3-ureidopropyltrialkoxysilane, 3-mercaptopropylmethyldimethoxysilane, 3-mercaptopropyltrimethoxysilane, 3-isocyanatepropyltriethoxysilane, and 3-trimethoxysilylpropyl succinic anhydride.

The silica particles surface-treated with a silane compound or a silane coupling agent may be further hydrophobicized by a surface treatment using a silane compound such as 3-methacryloxypropyltrimethoxysilane, vinyltrimethoxysilane, or trimethoxyphenylsilane, in order to further hydrophobicize the hydroxyl residue on the surface of the silica particles.

In the case of using the adhesive composition as an adhesive film for circuit connection, when the adhesive film for circuit connection is press-bonded, from the viewpoint of easily controlling fluidity and the viewpoint of improving the mechanical physical properties and water resistance of the connected structure after press-bonding, the surface-treated silica particles may contain at least one selected from the group consisting of a reaction product (hydrolysate product) of silica and trimethoxyoctylsilane, a reaction product of silica and dimethylsiloxane, a reaction product of silicon dioxide or silica and dichloro(dimethyl)silane, a reaction product (hydrolysate product) of silica and bis(trimethylsilyl)amine, and a reaction product of silica and hexamethyldisilazane and may contain at least one selected from the group consisting of a reaction product of silica and trimethoxyoctylsilane and a reaction product of silica and bis(trimethylsilyl)amine.

The content of the filler may be 5% by mass or more, 10% by mass or more, or 15% by mass or more, based on the total mass of the adhesive composition. The content of the filler may be 50% by mass or less, 30% by mass or less, or 20% by mass or less, based on the total mass of the adhesive composition.

The content of the filler may be 10 parts by mass or more, 25 parts by mass or more, or 40 parts by mass or more, based on 100 parts by mass of the cationic polymerizable compound. The content of the filler may be 100 parts by mass or less, 60 parts by mass or less, or 40 parts by mass or less, based on 100 parts by mass of the cationic polymerizable compound.

The adhesive composition may further contain components other than the above-described components. As the other components, the adhesive composition may contain a thermoplastic resin, a coupling agent, a filler, a stabilizer, a colorant, an antioxidant, a curing agent other than the curing agent containing the pyridinium salt A, and the like. The adhesive composition may further contain a radical polymerizable compound and a radical polymerization initiator.

Examples of the radical polymerizable compound include acrylic compounds. Examples of the acrylic compounds include a (meth)acrylic acid compound, a (meth)acrylate compound, and an imide compound of these. These may be used in either monomer or oligomer state, and a monomer and an oligomer may be used in combination. The radical polymerizable compound may be used singly or may be used in combination of two or more kinds thereof.

Specific examples of the acrylic compounds include alkyl (meth)acrylate compounds such as methyl acrylate, ethyl acrylate, isopropyl acrylate, and isobutyl acrylate; polyol poly(meth)acrylate compounds such as ethylene glycol diacrylate, diethylene glycol diacrylate, trimethylol propane triacrylate, and tetramethylol methane tetraacrylate; aryloxy-hydroxyalkyl (meth)acrylate compounds such as 2-hydroxy-1,3-diacryloxypropane, 2,2-bis[4-(acryloxymethoxy)phenyl]propane, and 2,2-bis[4-(acryloxypolyethoxy)phenyl]propane; and dicyclopentenyl acrylate, tricyclodecanyl acrylate, and tris(acryloyloxyethyl)isocyanurate.

The radical polymerization initiator may be one that generates free radicals by light or heat. Examples of the radical polymerization initiator include an organic peroxide and an azo-based compound. Examples of the organic peroxide include peroxyester, dialkyl peroxide, diacyl peroxide, peroxydicarbonate, peroxyketal, hydroperoxide, and silyl peroxide. The radical polymerization initiator may be used singly or may be used in combination of two or more kinds thereof.

Examples of the peroxyester include cumyl peroxyneodecanoate, 1,1,3,3-tetramethylbutyl peroxyneodecanoate, 1-cyclohexyl-1-methylethyl peroxyneodecanoate, t-hexyl peroxyneodecanoate, t-butyl peroxypivalate, 1,1,3,3-tetramethylbutylperoxy-2-ethyl hexanonate, 2,5-dimethyl-2,5-di(2-ethylhexanoylperoxy)hexane, 1-cyclohexyl-1-methylethylperoxy-2-ethyl hexanonate, L-hexylperoxy-2-ethyl hexanonate, L-butylperoxy-2-ethyl hexanonate, t-butylperoxyisobutyrate, 1,1-bis(t-butylperoxy)cyclohexane, t-hexylperoxy isopropyl monocarbonate, t-butylperoxy-3,5,5-trimethylhexanonate, t-butylperoxy laurate, 2,5-dimethyl-2,5-di(m-toluoylperoxy)hexane, t-butylperoxy isopropyl monocarbonate, t-butylperoxy-2-ethylhexyl monocarbonate, t-hexylperoxy benzoate, and t-butylperoxyacetate.

Examples of the dialkyl peroxide include α,α′-bis(t-butylperoxy)diisopropylbenzene, dicumyl peroxide, 2,5-dimethyl-2,5-di(t-butylperoxy)hexane, and t-butyl cumyl peroxide. Examples of the hydroperoxide include diisopropylbenzene hydroperoxide and cumene hydroperoxide.

Examples of the diacyl peroxide include isobutyl peroxide, 2,4-dichlorobenzoyl peroxide, 3,5,5-trimethylhexanoyl peroxide, octanoyl peroxide, lauroyl peroxide, stearoyl peroxide, succinic peroxide, benzoyl peroxytoluene, and benzoyl peroxide.

Examples of the peroxydicarbonate include di-n-propyl peroxydicarbonate, diisopropyl peroxydicarbonate, bis(4-t-butylcyclohexyl) peroxydicarbonate, di-2-ethoxymethoxy peroxydicarbonate, di(2-ethylhexylperoxy)dicarbonate, dimethoxybutyl peroxydicarbonate, and di(3-methyl-3-methoxybutylperoxy)dicarbonate.

Specific examples of the peroxyketal include 1,1-bis(t-hexylperoxy)-3,3,5-trimethylcyclohexane, 1,1-bis(t-hexylperoxy)cyclohexane, 1,1-bis(t-butylperoxy)-3,3,5-trimethylcyclohexane, 1, 1-(t-butylperoxy)cyclododecane, and 2,2-bis(t-butylperoxy)decane.

Specific examples of the silyl peroxide include t-butyl trimethylsilyl peroxide, bis(t-butyl)dimethylsilyl peroxide, t-butyl trivinylsilyl peroxide, bis(t-butyl)divinylsilyl peroxide, tris(t-butyl)vinylsilyl peroxide, t-butyl triallylsilyl peroxide, bis(t-butyl)diallylsilyl peroxide, and tris(t-butyl)allylsilyl peroxide.

### <Adhesive Film for Circuit Connection>

The adhesive composition may have a film shape. That is, still another embodiment of the present disclosure is an adhesive film for circuit connection, containing a pyridinium salt A and a cationic polymerizable compound. Furthermore, still another embodiment of the present disclosure is an adhesive film for circuit connection, containing a curing agent containing a pyridinium salt A and a cationic polymerizable compound. The adhesive film for circuit connection may contain conductive particles.

The particle density of the conductive particles in the adhesive film for circuit connection may be 100/mm² or more, 1000/mm² or more, or 3000/mm² or more, from the viewpoint of obtaining stable connection resistance. The particle density of the conductive particles in the adhesive film for circuit connection may be 100000/mm² or less, 50000/mm² or less, or 30000/mm² or less, from the viewpoint of ensuring the insulating property between adjacent electrodes. From these viewpoints, the particle density of the conductive particles in the adhesive film for circuit connection may be 100 to 100000/mm², 1000 to 50000/mm², or 3000 to 30000/mm².

The content of the conductive particles may be 10% by mass or more, 20% by mass or more, or 25% by mass or more, based on the total mass of the adhesive film for circuit connection. The content of the conductive particles may be 50% by mass or less, 40% by mass or less, or 30% by mass or less, based on the total mass of the adhesive film for circuit connection.

The content of the conductive particles may be 10 parts by mass or more, 30 parts by mass or more, 50 parts by mass or more, 70 parts by mass or more, or 90 parts by mass or more, based on 100 parts by mass of the cationic polymerizable compound. The content of the conductive particles may be 200 parts by mass or less, 150 parts by mass or less, 120 parts by mass or less, or 100 parts by mass or less, based on 100 parts by mass of the cationic polymerizable compound.

The content of the curing agent containing the pyridinium salt A in the adhesive film for circuit connection may be 1% by mass or more, 2% by mass or more, 3% by mass or more, 4% by mass or more, 4.5% by mass or more, 5% by mass or more, 6% by mass or more, 7% by mass or more, 8% by mass or more, 10% by mass or more, 11% by mass or more, or 11.5% by mass or more, based on the total mass of the adhesive film for circuit connection, from the viewpoint of sufficiently promoting a curing reaction. The content of the curing agent containing the pyridinium salt A in the adhesive film for circuit connection may be 20% by mass or less, 15% by mass or less, 12% by mass or less, 10% by mass or less, 9% by mass or less, 8% by mass or less, 7% by mass or less, 6% by mass or less, 5% by mass or less, or 4% by mass or less, based on the total mass of the adhesive film for circuit connection, from the viewpoint of improving the physical properties of a cured product. From these viewpoints, the content of the curing agent containing the pyridinium salt A in the adhesive film for circuit connection may be 1 to 20% by mass or 1 to 15% by mass based on the total mass of the adhesive film for circuit connection. The content of the pyridinium salt A in the adhesive film for circuit connection may be within the above content range.

The content of the curing agent containing the pyridinium salt A in the adhesive film for circuit connection may be 1% by mass or more, 2% by mass or more, 3% by mass or more, 4% by mass or more, 4.5% by mass or more, 5% by mass or more, 6% by mass or more, 8% by mass or more, 9% by mass or more, 10% by mass or more, 11% by mass or more, 11.5% by mass or more, 12% by mass or more, or 13% by mass or more, based on the total mass of the adhesive film for circuit connection excluding conductive particles, from the viewpoint of sufficiently promoting a curing reaction. The content of the curing agent containing the pyridinium salt A in the adhesive film for circuit connection may be 30% by mass or less, 25% by mass or less, 20% by mass or less, 15% by mass or less, 14% by mass or less, 12% by mass or less, 10% by mass or less, 9% by mass or less, 8% by mass or less, 7% by mass or less, 6% by mass or less, 5% by mass or less, 4% by mass or less, or 3% by mass or less, based on the total mass of the adhesive film for circuit connection excluding conductive particles, from the viewpoint of improving the physical properties of a cured product. From these viewpoints, the content of the curing agent containing the pyridinium salt A in the adhesive film for circuit connection may be 1 to 30% by mass based on the total mass of the adhesive film for circuit connection excluding conductive particles. The content of the pyridinium salt A in the adhesive film for circuit connection may be within the above content range.

The content of the curing agent containing the pyridinium salt A in the adhesive film for circuit connection may be 1% by mass or more, 3% by mass or more, 4% by mass or more, 4.5% by mass or more, 5% by mass or more, 5.5% by mass or more, 6% by mass or more, 7% by mass or more, 8% by mass or more, 10% by mass or more, 11% by mass or more, 11.5% by mass or more, 12% by mass or more, or 14% by mass or more, based on the total mass of the adhesive film for circuit connection excluding conductive particles and a filler, from the viewpoint of sufficiently promoting a curing reaction. The content of the curing agent containing the pyridinium salt A in the adhesive film for circuit connection may be 30% by mass or less, 25% by mass or less, 20% by mass or less, 15% by mass or less, 12% by mass or less, 11% by mass or less, 10% by mass or less, 9% by mass or less, 8% by mass or less, 7% by mass or less, 6% by mass or less, 5% by mass or less, 4% by mass or less, or 3% by mass or less, based on the total mass of the adhesive film for circuit connection excluding conductive particles and a filler, from the viewpoint of improving the physical properties of a cured product. From these viewpoints, the content of the curing agent containing the pyridinium salt A in the adhesive film for circuit connection may be 1 to 30% by mass based on the total mass of the adhesive film for circuit connection excluding conductive particles and a filler. The content of the pyridinium salt A in the adhesive film for circuit connection may be within the above content range.

The content of the cationic polymerizable compound in the adhesive film for circuit connection may be 10% by mass or more, 20% by mass or more, 25% by mass or more, or 30% by mass or more, based on the total mass of the adhesive film for circuit connection, from the viewpoint of securing the curability of the adhesive film for circuit connection. The content of the cationic polymerizable compound in the adhesive film for circuit connection may be 70% by mass or less, 50% by mass or less, 40% by mass or less, or 35% by mass or less, based on the total mass of the adhesive film for circuit connection, from the viewpoint of securing the formability of the adhesive film for circuit connection. From these viewpoints, the content of the cationic polymerizable compound in the adhesive film for circuit connection may be 10 to 70% by mass based on the total mass of the adhesive film for circuit connection.

The content of the thermoplastic resin in the adhesive film for circuit connection may be 5% by mass or more, 10% by mass or more, or 15% by mass or more, based on the total mass of the adhesive film for circuit connection. The content of the thermoplastic resin in the adhesive film for circuit connection may be 40% by mass or less, 30% by mass or less, or 20% by mass or less, based on the total mass of the adhesive film for circuit connection.

The content of the coupling agent in the adhesive film for circuit connection may be 0.5% by mass or more, 1% by mass or more, or 1.5% by mass or more, based on the total mass of the adhesive film for circuit connection. The content of the coupling agent in the adhesive film for circuit connection may be 10% by mass or less, 5% by mass or less, or 2% by mass or less, based on the total mass of the adhesive film for circuit connection.

The content of the filler in the adhesive film for circuit connection may be 5% by mass or more, 10% by mass or more, or 12% by mass or more, based on the total mass of the adhesive film for circuit connection. The content of the filler in the adhesive film for circuit connection may be 30% by mass or less, 20% by mass or less, or 15% by mass or less, based on the total mass of the adhesive film for circuit connection.

The content of each component in the adhesive film for circuit connection based on 100 parts by mass of the cationic polymerizable compound may be within the same range as the content of each component in the adhesive composition based on 100 parts by mass of the cationic polymerizable compound.

The adhesive film for circuit connection may be a single layer, and may have a multi-layer structure in which a plurality of layers are laminated. In a case where the adhesive film for circuit connection has a multi-layer structure, the adhesive film for circuit connection may include, for example, a first adhesive layer containing a pyridinium salt A (or a curing agent containing a pyridinium salt A) and a cationic polymerizable compound and a second adhesive layer other than the first adhesive layer. That is, the adhesive film for circuit connection may include a first adhesive layer and a second adhesive layer laminated on the first adhesive layer. At least one of the first adhesive layer and the second adhesive layer may contain a pyridinium salt A (or a curing agent containing a pyridinium salt A), a cationic polymerizable compound, and conductive particles. In a case where the adhesive film for circuit connection has a multi-layer structure, the content of each component described above in each layer may be within the above content range based on the total mass of each layer.

The adhesive film for circuit connection may have a plurality of regions having different types, contents, and the like of components. The adhesive film for circuit connection may include, for example, a first region and a second region disposed on the first region, and the first region may be a region containing a pyridinium salt A (or a curing agent containing a pyridinium salt A) and a cationic polymerizable compound. That is, in the adhesive film for circuit connection, a first region, which is a region formed from a first adhesive composition containing a pyridinium salt A (or a curing agent containing a pyridinium salt A) and a cationic polymerizable compound, and a second region, which is a region formed from a second adhesive composition and is disposed on the first region, may exist. In a case where the adhesive film for circuit connection has a plurality of regions, the content of each component described above in each region may be within the above content range based on the total mass of each region.

The adhesive film for circuit connection may be provided on a substrate (for example, a PET film) or the like. An adhesive film for circuit connection with a substrate can be produced, for example, by applying an adhesive composition containing conductive particles onto a substrate using a knife coater, a roll coater, an applicator, a comma coater, a die coater, or the like.

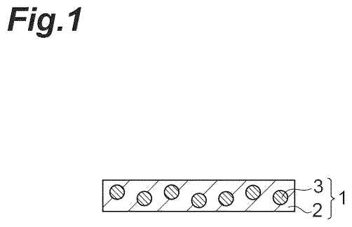

FIG. 1 is a schematic cross-sectional view illustrating an adhesive film for circuit connection according to an embodiment. As illustrated in FIG. 1, in an embodiment, an adhesive film 1 for circuit connection is configured by a single layer containing an adhesive component 2 and conductive particles 3 dispersed in the adhesive component 2. In an embodiment, the adhesive component 2 contains at least a pyridinium salt A (or a curing agent containing a pyridinium salt A) and a cationic polymerizable compound. The adhesive film 1 for circuit connection may be in an uncured state and may be in a partially cured state.

The thickness of the adhesive film 1 for circuit connection may be, for example, 3 μm or more or 10 μm or more and may be 30 μm or less or 20 μm or less.

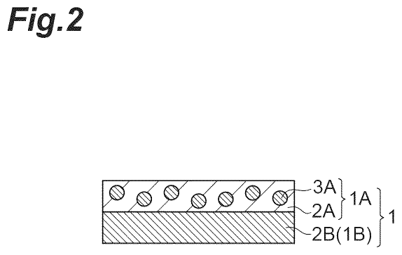

In an embodiment, the adhesive film for circuit connection may have a multi-layer structure having two or more layers, and for example, as illustrated in FIG. 2, the adhesive film 1 for circuit connection may have a two-layer structure which includes a layer 1A containing conductive particles 3A (a first adhesive layer composed of an adhesive component 2A and conductive particles 3A dispersed in the adhesive component 2A) and a layer 1B not containing conductive particles (a second adhesive layer composed of an adhesive component 2B). In this case, the first adhesive layer 1A may be a layer composed of an adhesive composition (first adhesive composition) containing a pyridinium salt A (or a curing agent containing a pyridinium salt A), a cationic polymerizable compound, and conductive particles. The second adhesive layer 1B may be a layer composed of an adhesive composition (second adhesive composition) containing a pyridinium salt A (or a curing agent containing a pyridinium salt A) and a cationic polymerizable compound. The type, content, and the like of each component contained in the second adhesive layer 1B may be the same as or different from those in the first adhesive layer 1A. Each of the first adhesive layer 1A and the second adhesive layer 1B of the adhesive film 1 for circuit connection may be in an uncured state and may be in a partially cured state.

The thickness of the first adhesive layer 1A may be, for example, 3 μm or more or 5 μm or more and may be 15 μm or less or 10 μm or less. The thickness of the second adhesive layer 1B may be, for example, 3 μm or more or 10 μm or more and may be 20 μm or less or 15 μm or less. The thickness of the first adhesive layer 1A may be the same as or different from the thickness of the second adhesive layer 1B. The ratio of the thickness of the first adhesive layer 1A and the thickness of the second adhesive layer 1B (the thickness of the first adhesive layer 1A/the thickness of the second adhesive layer 1B) may be 0.1 or more or 0.3 or more and may be 1.5 or less or 0.5 or less.

The adhesive film for circuit connection may be an anisotropic conductive adhesive film (anisotropic conductive film) and may be a conductive adhesive film not having anisotropic conductivity.

### <Connected Structure>

Still another embodiment of the present disclosure is a connected structure including a first circuit member having a first electrode, a second circuit member having a second electrode, and a connection portion disposed between the first circuit member and the second circuit member and electrically connecting the first electrode and the second electrode to each other, in which the connection portion includes a cured product of the above-described adhesive film for circuit connection.

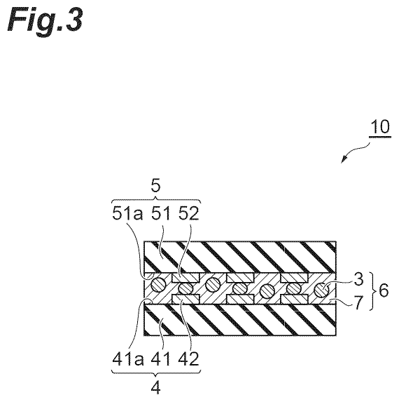

FIG. 3 is a schematic cross-sectional view illustrating an embodiment of a connected structure. As illustrated in FIG. 3, a structure 10 includes a first circuit member 4 and a second circuit member 5 facing each other, and a connection portion 6 connecting the first circuit member 4 and the second circuit member 5 between the first circuit member 4 and the second circuit member 5.

The first circuit member 4 includes a first circuit board 41 and a first electrode 42 formed on a main surface 41a of the first circuit board 41. The second circuit member 5 includes a second circuit board 51 and a second electrode 52 formed on a main surface 51a of the second circuit board 51.

The first circuit member 4 and the second circuit member 5 are not particularly limited as long as they are a member on which an electrode requiring electrical connection is formed. As the member (such as a circuit member) on which an electrode is formed, inorganic substrates such as semiconductors, glass, or ceramics; polyimide substrates typified by TCP, FPC, COF, and the like; substrates in which an electrode is formed on a film of polycarbonate, polyester, polyether sulfone, or the like; printed circuit boards; and the like are used, and a plurality of these members may be used in combination.

The connection portion 6 contains a cured product of the adhesive film 1 for circuit connection and contains an insulating material 7 that is a cured product of the adhesive component 2 and conductive particles 3. The conductive particles 3 may be disposed not only between the first electrode 42 and the second electrode 52 facing each other but also between the main surface 41a of the first circuit board 41 and the main surface 51a of the second circuit board 51. In the structure 30, the first electrode 42 and the second electrode 52 are electrically connected through the conductive particles 3. That is, the conductive particles 3 are in contact with both the first electrode 42 and the second electrode 52.

In the structure 10, as described above, the first electrode 42 and the second electrode 52 facing each other are electrically connected through the conductive particles 3. For this reason, the connection resistance between the first electrode 42 and the second electrode 52 is sufficiently reduced. Therefore, it is possible to achieve a smooth flow of current between the first electrode 42 and the second electrode 52, thereby allowing the function of the first circuit member 4 and the second circuit member 5 to be sufficiently exhibited.

### <Method for Producing Connected Structure>

Still another embodiment of the present disclosure is a method for producing a connected structure, the method including a step of interposing the above-described adhesive film for circuit connection between a first circuit member having a first electrode and a second circuit member having a second electrode, and thermocompression bonding the first circuit member and the second circuit member to electrically connect the first electrode and the second electrode to each other.

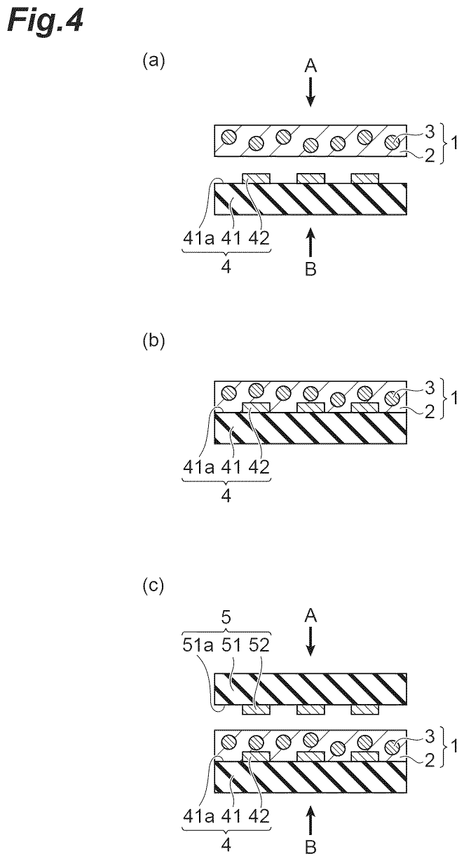

FIG. 4 is a schematic cross-sectional view illustrating an embodiment of a method for producing the connected structure. As illustrated in FIG. 4(a), first, the first circuit member 4 and the adhesive film 1 for circuit connection are prepared. Then, the adhesive film 1 for circuit connection is disposed on the main surface 41a of the first circuit member 4. In a case where the adhesive film 1 for circuit connection is laminated on a substrate (not illustrated), the laminate is disposed on the first circuit member 4 such that the substrate on the side of the adhesive film 1 for circuit connection faces the first circuit member 4. In a case where the adhesive film 1 for circuit connection has the first adhesive layer 1A and the second adhesive layer 1B as illustrated in FIG. 2, from the viewpoint of improving the number of conductive particles trapped between electrodes facing each other, the adhesive film 1 for circuit connection is preferably disposed such that the side of the adhesive layer (first adhesive layer 1A) containing conductive particles is in contact with the main surface 41a of the first circuit member 4.

Then, the adhesive film 1 for circuit connection is pressurized in the directions of arrows A and B in FIG. 4(a) to temporarily connect the adhesive film 1 for circuit connection to the first circuit member 4 (see FIG. 4(b)). At this time, heating may be performed with pressurizing.

Subsequently, as illustrated in FIG. 4(c), the second circuit member 5 is further disposed on the adhesive film 1 for circuit connection disposed on the first circuit member 4 such that the second electrode 52 side faces the first circuit member 4 (that is, in a state where the first electrode 42 and the second electrode 52 are disposed to face each other, the adhesive film 1 for circuit connection is interposed between the first circuit member 4 and the second circuit member 5). In a case where the adhesive film 1 for circuit connection is laminated on a substrate (not illustrated), the second circuit member 5 is disposed on the adhesive film 1 for circuit connection after the substrate is peeled off.

Then, the adhesive film 1 for circuit connection is thermocompression bonded in the directions of arrows A and B in FIG. 4(c). Thereby, the adhesive film 1 for circuit connection is cured to perform main connection in which the first electrode 42 and the second electrode 52 are electrically connected to each other. As a result, the structure 10 as illustrated in FIG. 3 is obtained.

In the structure 10 obtained as described above, the conductive particles 3 can be brought into contact with both the first electrode 42 and the second electrode 52 facing each other, and the connection resistance between the first electrode 42 and the second electrode 52 can be sufficiently reduced.

By pressurizing the adhesive film 1 for circuit connection on heating, the adhesive component 2 is cured in a state where a distance between the first electrode 42 and the second electrode 52 is sufficiently decreased to form the insulating material 7, and the first circuit member 4 and the second circuit member 5 are firmly connected via the connection portion 6. Furthermore, in the structure 10, a state where an adhesive strength is sufficiently high is maintained for a long time. Therefore, in the structure 10, a temporal change in distance between the first electrode 42 and the second electrode 52 is sufficiently suppressed, and long-term reliability of electrical characteristics between the first electrode 42 and the second electrode 52 is excellent.

### Examples

Hereinafter, the present disclosure will be specifically described by means of Examples. However, the present disclosure is not limited only to the following Examples.

### <Production of Curing Agent>

### [Synthesis and Analysis of Curing Agent A1]

100 mL of acetonitrile and a stirrer chip were placed in a 300 mL Erlenmeyer flask and the flask was placed on a magnetic stirrer. 12.5 g of 2-cyanopyridine (120 mmol, manufactured by Tokyo Chemical Industry Co., Ltd.), 15.7 g of 4-methoxybenzyl chloride (100 mmol, manufactured by Tokyo Chemical Industry Co., Ltd.), and 18.0 g of sodium iodide (120 mmol, manufactured by Tokyo Chemical Industry Co., Ltd.) were added to the acetonitrile in the 300 mL Erlenmeyer flask and the mixture was reacted at room temperature (25° C.) for 24 hours to obtain crystals. The obtained crystals were filtered through a glass filter, and the crystals on the glass filter were washed with acetone and distilled water and then vacuum-dried to obtain 14.2 g of 2-cyano-1-(4-methoxybenzyl)pyridinium iodide (yield: 40%).

35 mL of dichloromethane and a stirrer chip were placed in a 200 mL Erlenmeyer flask and the flask was placed on a magnetic stirrer. 3.5 g (10 mmol) of the obtained 2-cyano-1-(4-methoxybenzyl)pyridinium iodide was added to the 200 mL Erlenmeyer flask and suspended in the dichloromethane in the 200 mL Erlenmeyer flask. 72 g of a sodium tetrakis(pentafluorophenyl)borate aqueous solution (solid content: 10%) (10.2 mmol, manufactured by NIPPON SHOKUBAI CO., LTD.) and 70 mL of distilled water were added to the 200 mL Erlenmeyer flask, and the mixture was stirred at room temperature (25° C.) for 3 hours to perform a salt-exchange reaction. After stirring, the organic layer was washed with distilled water, concentrated, and vacuum-dried to obtain 7.9 g of a compound (yield: 87%). The obtained compound was regarded as a curing agent A1.

The obtained compound was measured by nuclear magnetic resonance spectrum (¹H-NMR, manufactured by JEOL Ltd., JNM-ECX400II) to obtain the following spectrum data. From the measurement by ¹H-NMR the obtained compound was confirmed to be 2-cyano-1-(4-methoxybenzyl)pyridinium tetrakis(pentafluorophenyl)borate having the following structure.

¹H-NMR (400 MHz, CD₃OD), δ: 3.81 (s, 3H), 6.02 (s, 2H), 7.03 (d, 2H, J=8.8 Hz), 7.47 (d, 2H, J=8.8 Hz), 8.38 (td, 1H, J=7.2, 1.6 Hz), 8.71 (dd, 1H, J=1, 2, 7.6 Hz), 8.80 (td, 1H, J=7.6, 1.2 Hz), 9.20 (d, 1H, J=5.6 Hz)

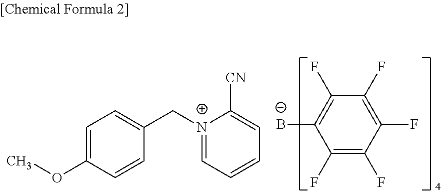

### [Synthesis and Analysis of Curing Agent A2]

100 mL of acetonitrile and a stirrer chip were placed in a 300 mL Erlenmeyer flask and the flask was placed on a magnetic stirrer. 12.5 g of 2-cyanopyridine (120 mmol, manufactured by Tokyo Chemical Industry Co., Ltd.), 16.8 g of 2,4,6-trimethylbenzyl chloride (100 mmol, manufactured by Tokyo Chemical Industry Co., Ltd.), and 17.8 g of sodium iodide (119 mmol, manufactured by Tokyo Chemical Industry Co., Ltd.) were added to the acetonitrile in the 300 mL Erlenmeyer flask and the mixture was reacted at room temperature (25° C.) for 24 hours to obtain crystals. The obtained crystals were filtered through a glass filter, and the crystals on the glass filter were washed with acetone and distilled water and then vacuum-dried to obtain 29.1 g of 2-cyano-1-(2,4,6-trimethylbenzyl)pyridinium iodide (yield: 80%).

200 mL of dichloromethane and a stirrer chip were placed in a 500 mL Erlenmeyer flask and the flask was placed on a magnetic stirrer. 3.6 g (10 mmol) of the obtained 2-cyano-1-(2,4,6-trimethylbenzyl)pyridinium iodide was added to the 500 mL Erlenmeyer flask and suspended in the dichloromethane in the 500 mL Erlenmeyer flask. 72 g of a sodium tetrakis(pentafluorophenyl)borate aqueous solution (solid content: 10%) (10.2 mmol, manufactured by NIPPON SHOKUBAI CO., LTD.) and 50 mL of distilled water were added to the 500 mL Erlenmeyer flask, and the mixture was stirred at room temperature (25° C.) for 3 hours to perform a salt-exchange reaction. After stirring, the organic layer was washed with distilled water, concentrated, and vacuum-dried to obtain 8.0 g of a compound (yield: 88%). The obtained compound was regarded as a curing agent A2.

The obtained compound was measured by nuclear magnetic resonance spectrum (¹H-NMR, manufactured by JEOL Ltd., JNM-ECX400II) to obtain the following spectrum data. From the measurement by ¹H-NMR the obtained compound was confirmed to be 2-cyano-1-(2,4,6-trimethylbenzyl)pyridinium tetrakis(pentafluorophenyl)borate having the following structure.

¹H-NMR (400 MHz, CD₃OD), δ: 2.26 (s, 6H), 2.32 (s, 3H), 6.10 (s, 2H), 7.08 (s, 2H), 8.25 (td, 1H, J=3.2, 6.4 Hz) 8.43 (d, 1H, J=6.4 Hz) 8.77-8.82 (m, 2H)

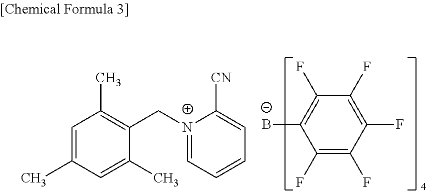

### [Synthesis and Analysis of Curing Agent A3]

100 mL of acetonitrile and a stirrer chip were placed in a 300 mL Erlenmeyer flask and the flask was placed on a magnetic stirrer. 12.5 g of 3-cyanopyridine (120 mmol, manufactured by Tokyo Chemical Industry Co., Ltd.), 16.8 g of 2,4,6-trimethylbenzyl chloride (100 mmol, manufactured by Tokyo Chemical Industry Co., Ltd.), and 17.8 g of sodium iodide (119 mmol, manufactured by Tokyo Chemical Industry Co., Ltd.) were added to the acetonitrile in the 300 mL Erlenmeyer flask and the mixture was reacted at room temperature (25° C.) for 24 hours to obtain crystals. The obtained crystals were filtered through a glass filter, and the crystals on the glass filter were washed with acetone and distilled water and then vacuum-dried to obtain 32.1 g of 3-cyano-1-(2,4,6-trimethylbenzyl)pyridinium iodide (yield: 88%).

36 mL of dichloromethane and a stirrer chip were placed in a 200 mL Erlenmeyer flask and the flask was placed on a magnetic stirrer. 3.6 g (10 mmol) of the obtained 3-cyano-1-(2,4,6-trimethylbenzyl)pyridinium iodide was added to the 200 mL Erlenmeyer flask and suspended in the dichloromethane in the 200 mL Erlenmeyer flask. 72 g of a sodium tetrakis(pentafluorophenyl)borate aqueous solution (solid content: 10%) (10.2 mmol, manufactured by NIPPON SHOKUBAI CO., LTD.) and 36 mL of distilled water were added to the 200 mL Erlenmeyer flask, and the mixture was stirred at room temperature (25° C.) for 3 hours to perform a salt-exchange reaction. After stirring, the organic layer was washed with distilled water, concentrated, and vacuum-dried to obtain 8.2 g of a compound (yield: 90%). The obtained compound was regarded as a curing agent A3.

The obtained compound was measured by nuclear magnetic resonance spectrum (¹H-NMR, manufactured by JEOL Ltd., JNM-ECX400II) to obtain the following spectrum data. From the measurement by ¹H-NMR, the obtained compound was confirmed to be 3-cyano-1-(2,4,6-trimethylbenzyl)pyridinium tetrakis(pentafluorophenyl)borate having the following structure.

¹H-NMR (400 MHz, CD₃OD), δ: 2.27 (s, 6H), 2.31 (s, 3H), 5.97 (s, 2H), 7.06 (s, 2H), 8.22 (t, 1H, J=6.8 Hz), 8.81 (d, 1H, J=6.0 Hz), 8.98 (d, 1H, J=7.6 Hz), 9.44 (s, 1H)

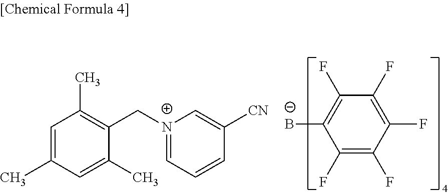

### [Synthesis and Analysis of Curing Agent A4]

100 mL of acetonitrile and a stirrer chip were placed in a 300 mL Erlenmeyer flask and the flask was placed on a magnetic stirrer. 12.5 g of 4-cyanopyridine (120 mmol, manufactured by Tokyo Chemical Industry Co., Ltd.), 16.8 g of 2,4,6-trimethylbenzyl chloride (100 mmol, manufactured by Tokyo Chemical Industry Co., Ltd.), and 17.8 g of sodium iodide (119 mmol, manufactured by Tokyo Chemical Industry Co., Ltd.) were added to the acetonitrile in the 300 mL Erlenmeyer flask and the mixture was reacted at room temperature (25° C.) for 24 hours to obtain crystals. The obtained crystals were filtered through a glass filter, and the crystals on the glass filter were washed with acetone and distilled water and then vacuum-dried to obtain 29.1 g of 4-cyano-1-(2,4,6-trimethylbenzyl)pyridinium iodide (yield: 80%).

36 mL of dichloromethane and a stirrer chip were placed in a 200 mL Erlenmeyer flask and the flask was placed on a magnetic stirrer. 3.6 g (10 mmol) of the obtained 4-cyano-1-(2,4,6-trimethylbenzyl)pyridinium iodide was added to the 200 mL Erlenmeyer flask and suspended in the dichloromethane in the 200 mL Erlenmeyer flask. 72 g of a sodium tetrakis(pentafluorophenyl)borate aqueous solution (solid content: 10%) (10.2 mmol, manufactured by NIPPON SHOKUBAI CO., LTD.) and 36 mL of distilled water were added to the 200 mL Erlenmeyer flask, and the mixture was stirred at room temperature (25° C.) for 3 hours to perform a salt-exchange reaction. After stirring, the organic layer was washed with distilled water, concentrated, and vacuum-dried to obtain 8.5 g of a compound (yield: 93%). The obtained compound was regarded as a curing agent A4.

The obtained compound was measured by nuclear magnetic resonance spectrum (¹H-NMR, manufactured by JEOL Ltd., JNM-ECX400II) to obtain the following spectrum data. From the measurement by ¹H-NMR the obtained compound was confirmed to be 4-cyano-1-(2,4,6-trimethylbenzyl)pyridinium tetrakis(pentafluorophenyl)borate having the following structure.

¹H-NMR (400 MHz, CD₃OD), δ: 2.25 (s, 6H), 2.28 (s, 3H), 6.00 (s, 2H), 7.03 (s, 2H), 8.46 (d, 2H, J=6.0 Hz) 8.96 (d, 2H, J=7.2 Hz)

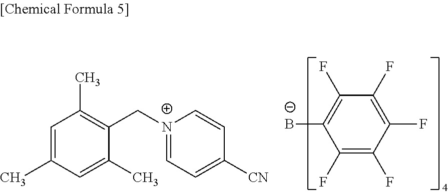

### [Synthesis and Analysis of Curing Agent A5]

100 mL of acetonitrile and a stirrer chip were placed in a 300 mL Erlenmeyer flask and the flask was placed on a magnetic stirrer. 14.5 g of N,N-dimethylaniline (120 mmol, manufactured by Tokyo Chemical Industry Co., Ltd.) and 15.6 g of 4-methoxybenzyl chloride (100 mmol, manufactured by Tokyo Chemical Industry Co., Ltd.) were added to the acetonitrile in the 300 mL Erlenmeyer flask and the mixture was reacted at room temperature (25° C.) for 24 hours to obtain crystals. The obtained crystals were filtered through a glass filter, and the crystals on the glass filter were washed with acetone and then vacuum-dried to obtain 16.6 g of N,N-dimethyl,N-(4-methoxybenzyl)anilinium chloride (yield: 60%).

36 mL of dichloromethane and a stirrer chip were placed in a 200 mL Erlenmeyer flask and the flask was placed on a magnetic stirrer. 2.8 g (10 mmol) of the obtained N,N-dimethyl,N-(4-methoxybenzyl)anilinium chloride was added to the 200 mL Erlenmeyer flask and suspended in the dichloromethane in the 200 mL Erlenmeyer flask. 72 g of a sodium tetrakis(pentafluorophenyl)borate aqueous solution (solid content: 10%) (10.2 mmol, manufactured by NIPPON SHOKUBAI CO., LTD.) and 36 mL of distilled water were added to the 200 mL Erlenmeyer flask, and the mixture was stirred at room temperature (25° C.) for 3 hours to perform a salt-exchange reaction. After stirring, the organic layer was washed with distilled water, concentrated, and vacuum-dried to obtain 7.8 g of a compound (yield: 85%). The obtained compound was regarded as a curing agent A5.

The obtained compound was measured by nuclear magnetic resonance spectrum (¹H-NMR, manufactured by JEOL Ltd., JNM-ECX400II) to obtain the following spectrum data. From the measurement by ¹H-NMR the obtained compound was confirmed to be N,N-dimethyl,N-(4-methoxybenzyl)anilinium tetrakis(pentafluorophenyl)borate having the following structure.

¹H-NMR (400 MHz, CD₃OD), δ: 3.60 (s, 6H), 3.75 (s, 3H), 4.92 (s, 2H), 6.83 (dt, 2H, J=8.8, 2.4 Hz), 6.97 (dt, 2H, J=8.8, 2.4 Hz), 7.58 to 7.63 (m, 3H), 7.69 to 7.73 (m, 2H)

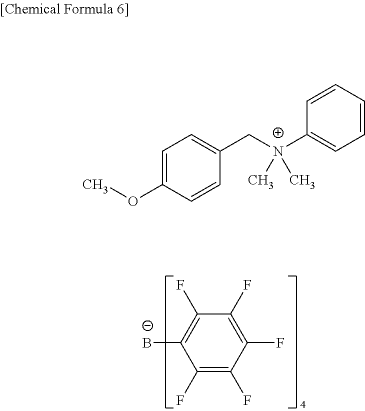

### [Synthesis and Analysis of Curing Agent A7]

100 mL of acetonitrile and a stirrer chip were placed in a 300 mL Erlenmeyer flask and the flask was placed on a magnetic stirrer. 12.5 g of 2-cyanopyridine (120 mmol, manufactured by Tokyo Chemical Industry Co., Ltd.), 12.6 g of benzyl chloride (100 mmol, manufactured by Tokyo Chemical Industry Co., Ltd.), and 17.8 g of sodium iodide (119 mmol, manufactured by Tokyo Chemical Industry Co., Ltd.) were added to the acetonitrile in the 300 mL Erlenmeyer flask, the mixture was reacted at room temperature (25° C.) for 24 hours, and then 100 ml of toluene was added to the mixture to obtain crystals. The obtained crystals were filtered through a glass filter, and the crystals on the glass filter were washed with toluene and then vacuum-dried to obtain a mixture of 2-cyano-1-(benzyl)pyridinium iodide, sodium chloride of by-product, and unreacted sodium iodide.

200 mL of dichloromethane and a stirrer chip were placed in a 500 mL Erlenmeyer flask and the flask was placed on a magnetic stirrer. The obtained mixture was added to the 500 mL Erlenmeyer flask and suspended in the dichloromethane in the 500 mL Erlenmeyer flask. 72 g of a sodium tetrakis(pentafluorophenyl)borate aqueous solution (solid content: 10%) (10.2 mmol, manufactured by NIPPON SHOKUBAI CO., LTD.) and 50 mL of distilled water were added to the 500 mL Erlenmeyer flask, and the mixture was stirred at room temperature (25° C.) for 3 hours to perform a salt-exchange reaction. After stirring, the organic layer was washed with distilled water, concentrated, and vacuum-dried to obtain 7.9 g of a compound (yield: 90%). The obtained compound was regarded as a curing agent A7.

The obtained compound was measured by nuclear magnetic resonance spectrum (¹H-NMR, manufactured by JEOL Ltd., JNM-ECX400II) to obtain the following spectrum data. From the measurement by ¹H-NMR the obtained compound was confirmed to be 2-cyano-1-(benzyl)pyridinium tetrakis(pentafluorophenyl)borate having the following structure.

¹H-NMR (400 MHz, CD₃OD), δ: 6.11 (s, 2H), 7.48 to 7.52 (m, 5H), 8.43 (ddd, 1H, J=8.0, 6.0, 1.2 Hz), 8.75 (dd, 1H, J=8.0, 1.2 Hz), 8.84 (td, 1H, J=8.8, 1.2 Hz), 9.31 (d, 1H, J=6.0 Hz)

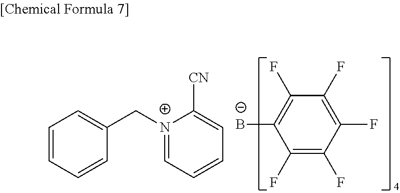

### [Synthesis and Analysis of Curing Agent A8]

100 mL of acetonitrile and a stirrer chip were placed in a 300 mL Erlenmeyer flask and the flask was placed on a magnetic stirrer. 12.5 g of 2-cyanopyridine (120 mmol, manufactured by Tokyo Chemical Industry Co., Ltd.), 16.1 g of 4-chlorobenzyl chloride (100 mmol, manufactured by Tokyo Chemical Industry Co., Ltd.), and 17.8 g of sodium iodide (119 mmol, manufactured by Tokyo Chemical Industry Co., Ltd.) were added to the acetonitrile in the 300 mL Erlenmeyer flask, the mixture was reacted at room temperature (25° C.) for 24 hours, and then 100 ml of toluene was added to the mixture to obtain crystals. The obtained crystals were filtered through a glass filter, and the crystals on the glass filter were washed with toluene and then vacuum-dried to obtain a mixture of 2-cyano-1-(4-chlorobenzyl)pyridinium iodide, sodium chloride of by-product, and unreacted sodium iodide.

200 mL of dichloromethane and a stirrer chip were placed in a 500 mL Erlenmeyer flask and the flask was placed on a magnetic stirrer. The obtained mixture was added to the 500 mL Erlenmeyer flask and suspended in the dichloromethane in the 500 mL Erlenmeyer flask. 72 g of a sodium tetrakis(pentafluorophenyl)borate aqueous solution (solid content: 10%) (10.2 mmol, manufactured by NIPPON SHOKUBAI CO., LTD.) and 50 mL of distilled water were added to the 500 mL Erlenmeyer flask, and the mixture was stirred at room temperature (25° C.) for 3 hours to perform a salt-exchange reaction. After stirring, the organic layer was washed with distilled water, concentrated, and vacuum-dried to obtain 8.2 g of a compound (yield: 90%). The obtained compound was regarded as a curing agent A8.

The obtained compound was measured by nuclear magnetic resonance spectrum (¹H-NMR, manufactured by JEOL Ltd., JNM-ECX400II) to obtain the following spectrum data. From the measurement by ¹H-NMR the obtained compound was confirmed to be 2-cyano-1-(4-chlorobenzyl)pyridinium tetrakis(pentafluorophenyl)borate having the following structure.

¹H-NMR (400 MHz, CD₃OD), δ: 6.11 (s, 2H), 7.50 to 7.55 (m, 4H), 8.43 (ddd, 1H, J=8.0, 6.0, 1.2 Hz), 8.75 (dd, 1H, J=8.0, 1.2 Hz), 8.85 (td, 1H, J=8.8, 1.2 Hz), 9.31 (d, 1H, J=6.0 Hz)

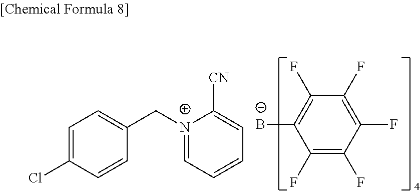

### [Synthesis and Analysis of Curing Agent A9]

100 mL of acetonitrile and a stirrer chip were placed in a 300 mL Erlenmeyer flask and the flask was placed on a magnetic stirrer. 12.5 g of 2-cyanopyridine (120 mmol, manufactured by Tokyo Chemical Industry Co., Ltd.), 17.7 g of 1-chloromethyl naphthalene (100 mmol, manufactured by Tokyo Chemical Industry Co., Ltd.), and 17.8 g of sodium iodide (119 mmol, manufactured by Tokyo Chemical Industry Co., Ltd.) were added to the acetonitrile in the 300 mL Erlenmeyer flask, the mixture was reacted at room temperature (25° C.) for 24 hours, and then 100 ml of toluene was added to the mixture to obtain crystals. The obtained crystals were filtered through a glass filter, and the crystals on the glass filter were washed with toluene and then vacuum-dried to obtain a mixture of 2-cyano-1-(naphthylmethyl)pyridinium iodide, sodium chloride of by-product, and unreacted sodium iodide.

200 mL of dichloromethane and a stirrer chip were placed in a 500 mL Erlenmeyer flask and the flask was placed on a magnetic stirrer. The obtained mixture was added to the 500 mL Erlenmeyer flask and suspended in the dichloromethane in the 500 mL Erlenmeyer flask. 72 g of a sodium tetrakis(pentafluorophenyl)borate aqueous solution (solid content: 10%) (10.2 mmol, manufactured by NIPPON SHOKUBAI CO., LTD.) and 50 mL of distilled water were added to the 500 mL Erlenmeyer flask, and the mixture was stirred at room temperature (25° C.) for 3 hours to perform a salt-exchange reaction. After stirring, the organic layer was washed with distilled water, concentrated, and vacuum-dried to obtain 8.3 g of a compound (yield: 90%). The obtained compound was regarded as a curing agent A9. The obtained compound was 2-cyano-1-(naphthylmethyl)pyridinium tetrakis(pentafluorophenyl)borate having the following structure.

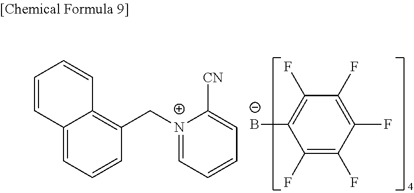

### <Production of Conductive Particles>

A layer composed of nickel was formed on the surface of cross-linked polystyrene particles so that the thickness of the layer was 0.15 μm. In this way, conductive particles having an average particle size of 3.3 μm, a maximum particle size of 3.5 μm, and a specific gravity of 2.7 were obtained.

### <Production of Adhesive Film for Circuit Connection>

Respective components were mixed in blended amounts (unit: parts by mass) shown in Tables 1 to 3 to prepare a first adhesive composition forming a first adhesive layer and a second adhesive composition forming a second adhesive layer. Note that, details of each component in Tables 1 to 3 are as follows, and the blended amount of each component in the table represents the blended amount of non-volatile content.

### Curing Agent

A1 to A5 and A7 to A9: Curing agent synthesized above
A6: 1-Naphthylmethylmethyl-p-hydroxyphenylsulfonium hexafluoroantimonate (SI-60, manufactured by SANSHIN CHEMICAL INDUSTRY CO., LTD.)

### Stabilizer

B: 4-Hydroxyphenyldimethylsulfonium sulfate (manufactured by Tokyo Chemical Industry Co., Ltd.)

### Cationic Polymerizable Compound

C1: Dicyclopentadiene dimethanol diglycidyl ether (trade name: EP-4088S, manufactured by ADEKA Corporation)
C2: Bisphenol A-type epoxy resin (trade name: YL980, manufactured by Mitsubishi Chemical Corporation)
C3: Tetramethylbiphenol-type epoxy resin (trade name: YX4000, manufactured by Mitsubishi Chemical Corporation)
C4: Xylene-novolac-type glycidyl ether (trade name: YX7700, manufactured by Mitsubishi Chemical Corporation)
C5: Solid-type bisphenol A epoxy resin (trade name: jER1010, manufactured by Mitsubishi Chemical Corporation)

### Thermoplastic Resin

D: Phenoxy resin a (synthetic)

### Coupling Agent

E: 3-Glycidoxypropyltrimethoxysilane (trade name: KBM-403, manufactured by Shin-Etsu Chemical Co., Ltd.)

### Filler

F1: Surface-treated silica fine particles (hydrolysate product of trimethoxyoctylsilane and silica, trade name: AEROSIL R805, manufactured by Evonik Industries AG, using those diluted with an organic solvent to have a non-volatile content of 10% by mass)
F2: Surface-treated silica particles (hydrolysate product of silica and bis(trimethylsilyl)amine)

### Conductive Particles

G: Conductive particles produced above
   <Synthesis of Phenoxy Resin a>

In a 3000 mL three-necked flask equipped with a Dimroth condenser, a calcium chloride tube, and a Teflon (registered trademark) stirring rod connected to a stirring motor, 45 g of 4,4′-(9-fluorenylidene)-diphenol (manufactured by Sigma-Aldrich Japan) and 50 g of 3,3′,5,5′-tetramethylbiphenol diglycidylether (trade name: YX-4000H, manufactured by Mitsubishi Chemical Corporation) were dissolved in 1000 mL of N-methylpyrrolidone to obtain a reaction solution. 21 g of potassium carbonate was added to this reaction solution and stirred for 3 hours on heating to 110° C. with a mantle heater. The reaction solution after stirring was added dropwise to a 1000 mL beaker containing methanol and a precipitate generated was filtered by suction filtration. The filtered precipitate was further washed with 300 mL of methanol three times to obtain 75 g of a phenoxy resin a. The molecular weight of the obtained phenoxy resin a was measured using a high-performance liquid chromatograph (manufactured by Tosoh Corporation, GP8020, column: Gelpack GL-A150S and GLA160S manufactured by Hitachi Chemical Company, Ltd., eluent: tetrahydrofuran, flow rate: 1.0 mL/min) and the molecular weight thereof was found to be Mn=15769, Mw=38045, and Mw/Mn=2.413 in terms of polystyrene.

The second adhesive composition was applied onto a substrate (PET film) to form the second adhesive layer on the substrate. Further, the first adhesive composition was applied onto the second adhesive layer to form the first adhesive layer, thereby producing an adhesive film for circuit connection in which the first adhesive layer, the second adhesive layer, and the substrate were laminated in this order. The thickness of the first adhesive layer of each adhesive film for circuit connection of Examples 1 to 10 and Comparative Examples 1 to 7 was 7 μm and the thickness of the second adhesive layer thereof was 11 μm.

### <Production of Connected Structure A>

As the first circuit member, one obtained by forming a wiring pattern (pattern width: 19 μm, space between electrodes: 5 μm) of AlNd (100 nm)/Mo (50 nm)/ITO (100 nm) on the surface of an alkali-free glass substrate (OA-11, manufactured by Nippon Electric Glass Co., Ltd., outer shape: 38 mm×28 mm, thickness: 0.3 mm) was prepared. As the second circuit member, an IC chip (outer shape: 0.9 mm×20.3 mm, thickness: 0.3 mm, size of bump electrode: 70 μm×12 μm, space between bump electrodes: 12 μm, bump electrode thickness: 8 μm) in which bump electrodes were arranged in two staggered rows was prepared.

A connected structure A was produced using each adhesive film for circuit connection of Examples 1 to 9 and Comparative Examples 1 to 7. First, the first adhesive layer of the adhesive film for circuit connection was disposed on the first circuit member. The adhesive film for circuit connection was bonded to the first circuit member by heating and pressurizing for 2 seconds under the conditions of 50° C. and 0.98 MPa (10 kgf/cm²) using a thermocompression bonding apparatus (manufactured by OHASHI ENGINEERING CO., LTD.) configured by a tool (8 mm×50 mm) and a stage including a ceramic heater. Then, the substrate on a side opposite to the first circuit member of the adhesive film for circuit connection was peeled off, and the bump electrode of the first circuit member and the circuit electrode of the second circuit member were aligned. Then, the second adhesive layer of the adhesive film for circuit connection was bonded to the second circuit member using a heat tool (8 mm×45 mm) by heating and pressurizing at 120° C. for 5 seconds and at 60 MPa on a base heated to 80° C. through a PTFE sheet having a thickness of 50 μm as a buffer material, thereby producing a connected structure A. In a case where mounting at 120° C. was not possible, a connected structure A was produced by raising the mounting temperature. Note that, the temperature was set to an actually measured highest arrival temperature of the adhesive film for circuit connection, and the pressure was set to a value calculated for the total area of the surface, which faces the first circuit member, of the bump electrode of the second circuit member.

### <Production of Connected Structure B>

A connected structure B was produced using each adhesive film for circuit connection of Examples 1 to 3 and Comparative Examples 1 to 5 in the same manner as in the connected structure A, except that one obtained by forming a wiring pattern (pattern width: 19 μm, space between electrodes: 5 μm) of Al alloy (200 nm)/Mo alloy (100 nm)/ITO (40 nm) on the surface of an alkali-free glass substrate (OA-11, manufactured by Nippon Electric Glass Co., Ltd., outer shape: 38 mm×28 mm, thickness: 0.5 mm) was used as the first circuit member, and the mounting temperature was set to 120° C. or 135° C.

### <Production of Connected Structure C>

A connected structure C was produced using each adhesive film for circuit connection of Examples 1 to 3 and Comparative Examples 1 to 5 in the same manner as in the connected structure A, except that one obtained by forming a wiring pattern (pattern width: 19 μm, space between electrodes: 5 μm) of ITO (300 nm) on the surface of an alkali-free glass substrate (OA-11, manufactured by Nippon Electric Glass Co., Ltd., outer shape: 38 mm×28 mm, thickness: 0.3 mm) was used as the first circuit member, and the mounting temperature was set to 120° C. or 135° C.

### <Evaluation of Connection Resistance>

The connection resistance at 14 places was measured using the connected structure A by a four-terminal measuring method, and maximum values (maximum resistance values) of connection resistance values immediately after connected structure production (initial stage) and after a high-temperature and high-humidity test were evaluated. The high-temperature and high-humidity test was performed by storing the connected structure in a thermostat-humidistat bath at a temperature of 85° C. and a humidity of 85% RH for 250 hours. In the measurement of the connection resistance, a multimeter (MLR21, manufactured by ETAC (Kusumoto Chemicals, Ltd.)) was used. The connection resistance was evaluated as follows: a case where the connection resistance was less than 1Ω was evaluated as “A”, a case where the connection resistance was 1Ω or more and less than 2Ω was evaluated as “B”, a case where the connection resistance was 2Ω or more and less than 5Ω was evaluated as “C”, a case where the connection resistance was 5Ω or more and less than 10Ω was evaluated as “D”, and a case where the connection resistance was 10Ω or more was evaluated as “E”. The evaluation results are shown in Tables 4 and 5.

### <Evaluation of Connection Resistance and Appearance>

The connection resistance at 14 places was measured using the connected structure B by a four-terminal measuring method, and maximum values (maximum resistance values) of connection resistance values after a high-speed degradation test were evaluated. The high-speed degradation test was performed by storing the connected structure in a thermostat-humidistat bath at a temperature of 110° C. and a humidity of 85% RH for 16 hours. The measurement and evaluation of the connection resistance were performed in the same manner as in the evaluation in the case of using the connected structure A. The appearance was evaluated as follows: a case where the peeling of the adhesive film for circuit connection was not confirmed was evaluated as “A”, a case where the area of the peeled portion was 30% or less was evaluated as “B”, and a case where the area of the peeled portion was more than 30% was evaluated as “C”. The evaluation results are shown in Table 6.

The connection resistance at 14 places was measured using the connected structure C by a four-terminal measuring method, and maximum values (maximum resistance values) of connection resistance values immediately after connected structure production (initial stage) and after a high-temperature and high-humidity test were evaluated. The high-temperature and high-humidity test and the measurement and evaluation of the connection resistance were performed in the same manner as in the evaluation in the case of using the connected structure A. The appearance was evaluated in the same manner as in the evaluation in the case of using the connected structure B. The evaluation results are shown in Table 6. In Table 6, “-” described for evaluation means “unmeasured”.

### <Bonding Property of Adhesive Film for Circuit Connection>

The adhesion when the adhesive film for circuit connection was bonded to the first circuit member of the connected structure C, and the peelability when the substrate was peeled off from the adhesive film for circuit connection were evaluated. The adhesion was evaluated as follows: when the adhesive film for circuit connection was bonded to the first circuit member, a case where pressurizing was not necessary was evaluated as “A”, a case where slight pressurizing was necessary was evaluated as “B”, a case where sufficient pressurizing was necessary was evaluated as “C”, and a case where the adhesive film for circuit connection was not bonded to the first circuit member even by pressurizing was evaluated as “D”. The peelability was evaluated as follows: when the substrate was peeled off from the adhesive film for circuit connection, a case where the substrate could be peeled off without the adhesive film for circuit connection being peeled off from the first circuit member was evaluated as “A”, and a case where the adhesive film for circuit connection was peeled off from the first circuit member was evaluated as “B”. The evaluation results are shown in Table 6.

### <Evaluation of Storage Stability>

After the adhesive film for circuit connection obtained in each of Examples and Comparative Examples was stored in a thermostat device set at 40° C. for 15 hours, the connected structure C was produced at a mounting temperature of 135° C. in the same manner as described above, and then the connection resistance (initial stage), the appearance (initial stage), and the adhesion were evaluated. The evaluation results are shown in Table 7.

### <DSC Measurement>

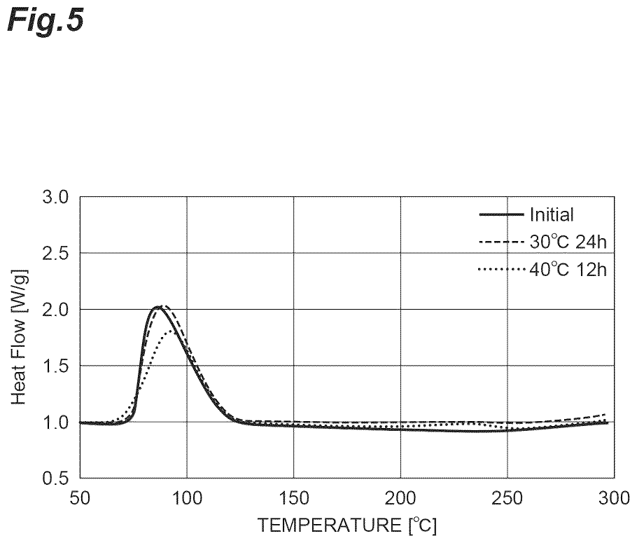

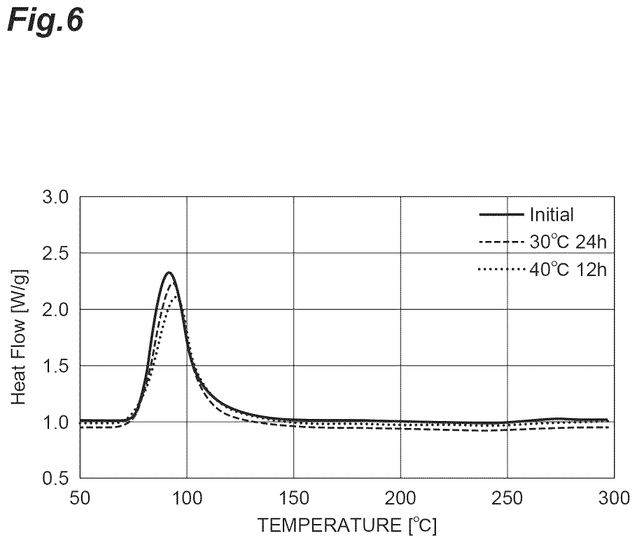

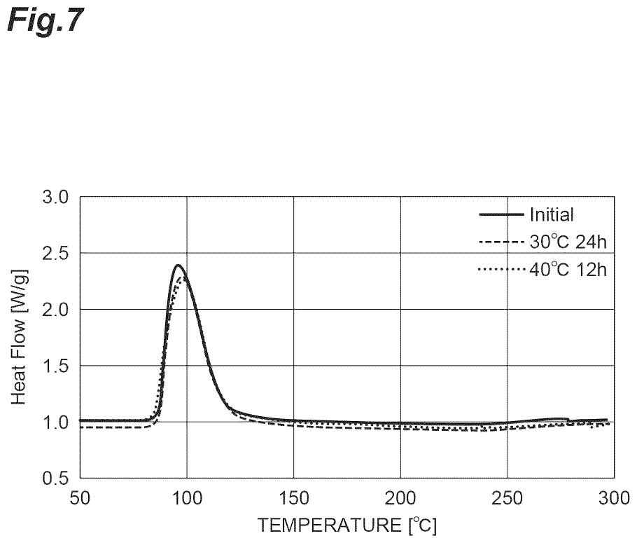

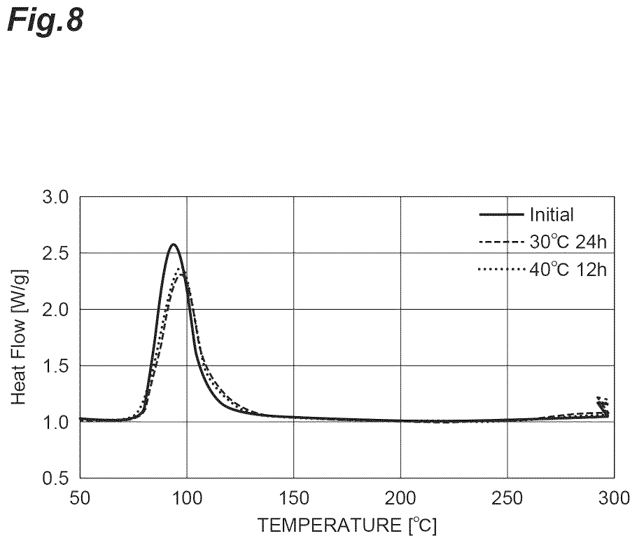

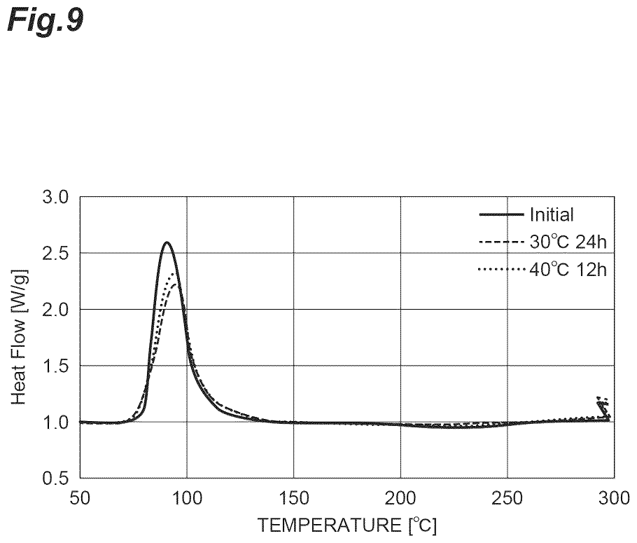

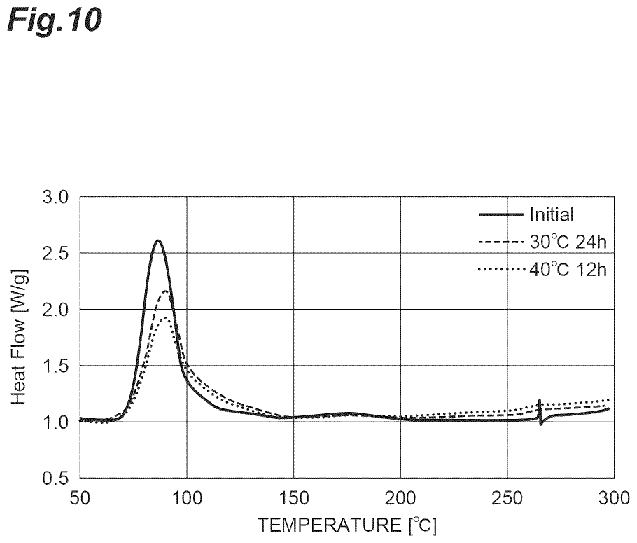

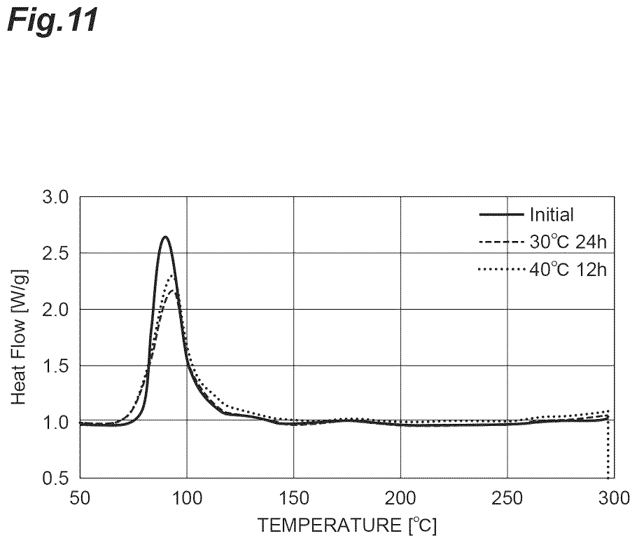

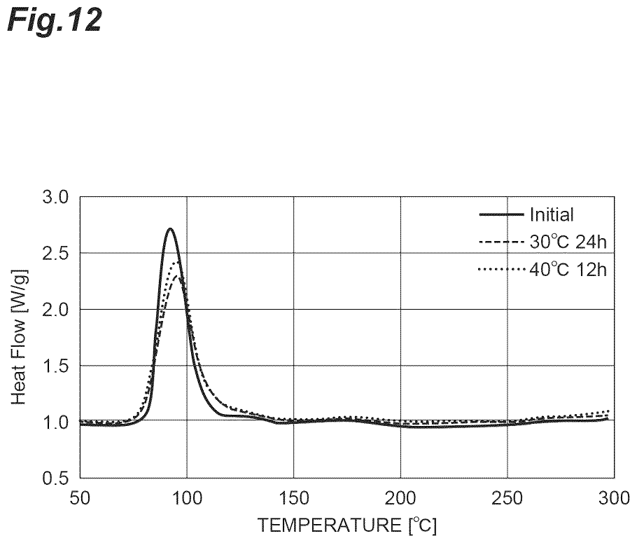

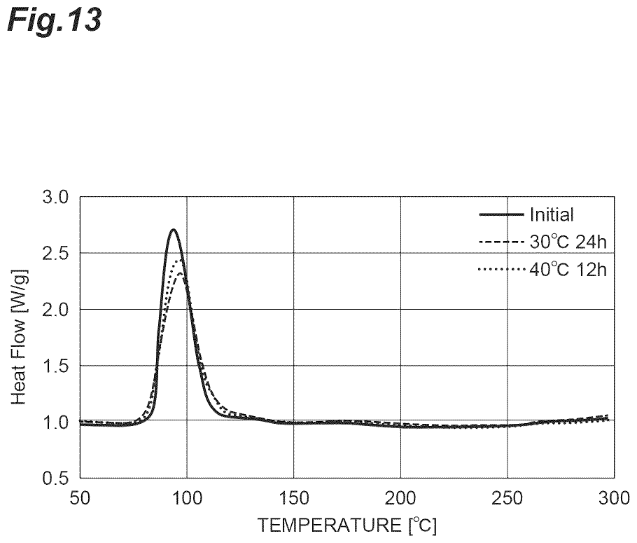

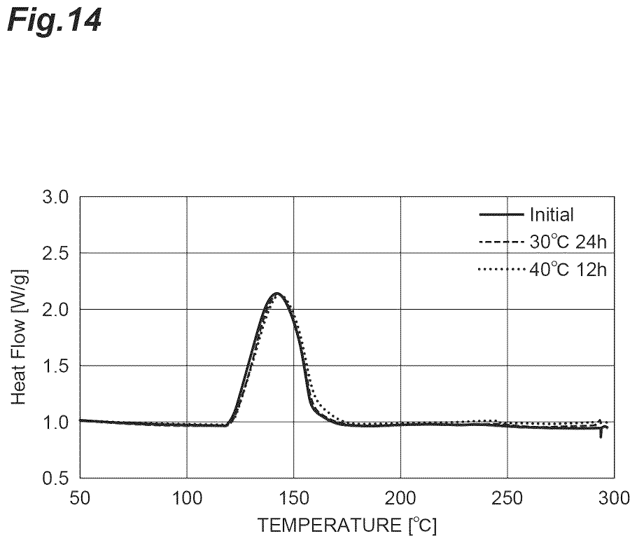

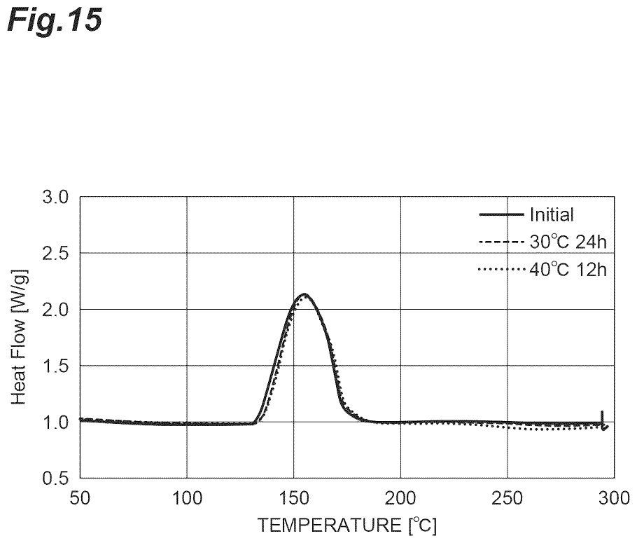

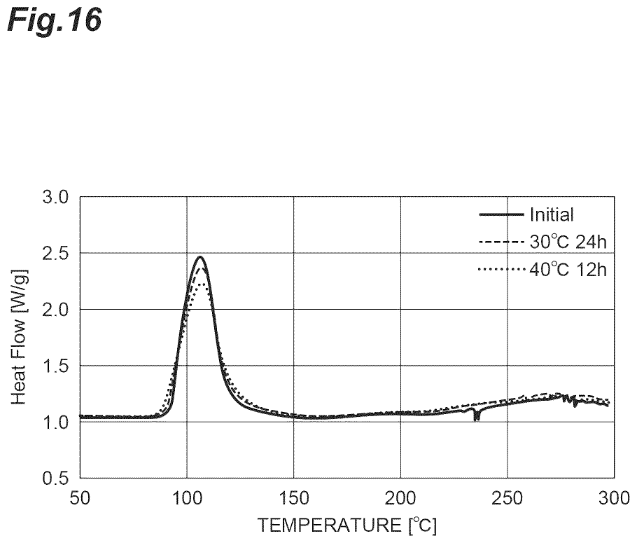

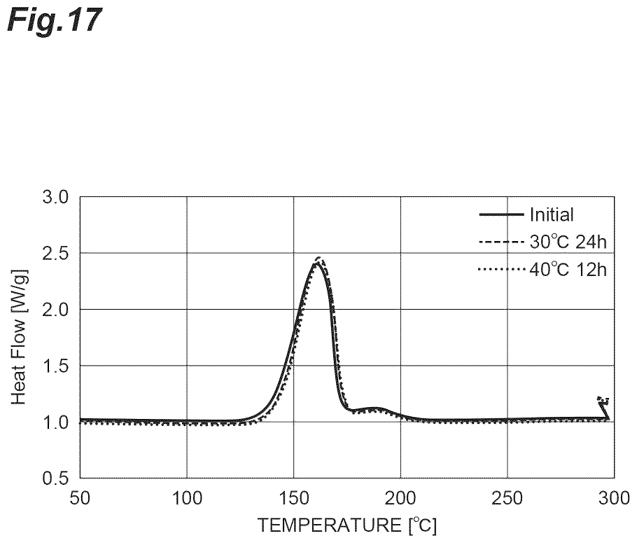

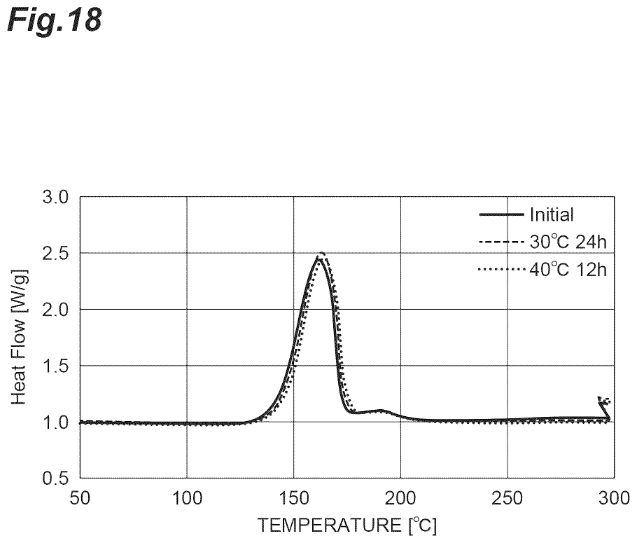

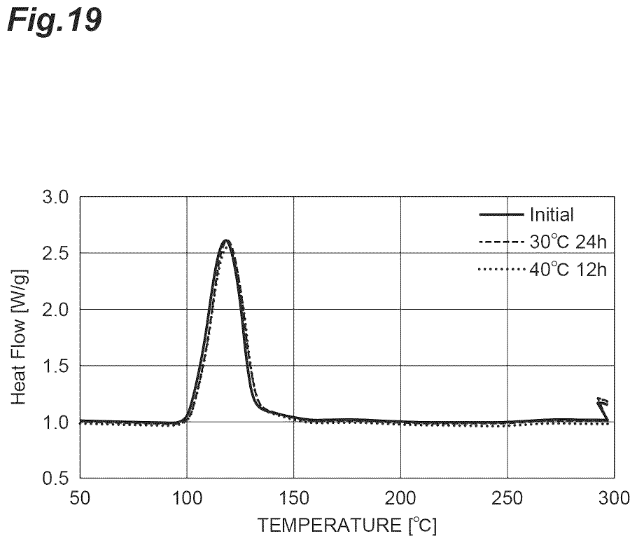

The adhesive films for circuit connection of Examples 1 to 9 and Comparative Examples 1 to 3 and 5 to 7 were subjected to differential scanning calorimetry (DSC) using a differential scanning calorimeter (trade name: DSC Q1000) manufactured by TA Instruments Japan Inc. in a nitrogen atmosphere under the conditions of a temperature increase rate of 10° C./min and a measurement temperature range of 50° C. to 300° C. The DSC measurement was performed with respect to a sample immediately after collection, a sample after storage at 30° C. for 24 hours, and a sample after storage at 40° C. for 12 hours. The DSC measurement results of each adhesive film for circuit connection are shown in FIGS. 5 to 19.

TABLE 1

| | Component | | Example 1 | Example 2 | Example 3 |
|---|---|---|---|---|---|
| First | Curing agent | A1 | 7 | — | — |
| adhesive | | A2 | — | 7 | 7 |
| layer | Stabilizer | B | 0.1 | — | 0.1 |
| | Cationic | C1 | 25 | 25 | 25 |
| | polymerizable | C2 | 25 | 25 | 25 |
| | compound | | | | |
| | Thermoplastic resin | D | 30 | 30 | 30 |
| | Coupling agent | E | 3 | 3 | 3 |
| | Filler | F1 | 5 | 5 | 5 |
| | | F2 | 15 | 15 | 15 |
| | Conductive particles | G | 45 | 45 | 45 |
| Second | Curing agent | A1 | 7 | — | — |
| adhesive | | A2 | — | 7 | 7 |
| layer | Stabilizer | B | 0.1 | — | 0.1 |
| | Cationic | C1 | 15 | 15 | 15 |
| | polymerizable | C3 | 15 | 15 | 15 |
| | compound | C4 | 10 | 10 | 10 |
| | | C5 | 30 | 30 | 30 |
| | Thermoplastic resin | D | 10 | 10 | 10 |
| | Coupling agent | E | 3 | 3 | 3 |
| | Filler | G2 | 20 | 20 | 20 |

TABLE 2

| | | | Example | Example | Example | Example | Example | Example |
|---|---|---|---|---|---|---|---|---|
| | Component | | 4 | 5 | 6 | 7 | 8 | 9 |
| First | Curing agent | A2 | 5 | 10 | 14 | 14 | 14 | 14 |
| adhesive | Stabilizer | B | — | 0.05 | — | 0.05 | 0.1 | 0.15 |
| layer | Cationic | C1 | 25 | 25 | 25 | 25 | 25 | 25 |
| | polymerizable | C2 | 25 | 25 | 25 | 25 | 25 | 25 |
| | compound | | | | | | | |
| | Thermoplastic resin | D | 30 | 30 | 30 | 30 | 30 | 30 |
| | Coupling agent | E | 3 | 3 | 3 | 3 | 3 | 3 |
| | Filler | F1 | 5 | 5 | 5 | 5 | 5 | 5 |
| | | F2 | 15 | 15 | 15 | 15 | 15 | 15 |
| | Conductive particles | G | 45 | 45 | 45 | 45 | 45 | 45 |
| Second | Curing agent | A2 | 5 | 10 | 14 | 14 | 14 | 14 |
| adhesive | Stabilizer | B | — | 0.05 | — | 0.05 | 0.1 | 0.15 |
| layer | Cationic | C1 | 15 | 15 | 15 | 15 | 15 | 15 |
| | polymerizable | C3 | 15 | 15 | 15 | 15 | 15 | 15 |
| | compound | C4 | 10 | 10 | 10 | 10 | 10 | 10 |
| | | C5 | 30 | 30 | 30 | 30 | 30 | 30 |
| | Thermoplastic resin | D | 10 | 10 | 10 | 10 | 10 | 10 |
| | Coupling agent | E | 3 | 3 | 3 | 3 | 3 | 3 |
| | Filler | G2 | 20 | 20 | 20 | 20 | 20 | 20 |

TABLE 3

| | | | Comparative | Comparative | Comparative | Comparative | Comparative | Comparative | Comparative |
|---|---|---|---|---|---|---|---|---|---|
| | Component | | Example 1 | Example 2 | Example 3 | Example 4 | Example 5 | Example 6 | Example 7 |
| First | Curing agent | A3 | 7 | — | — | — | — | — | — |
| adhesive | | A4 | — | 7 | — | — | — | — | — |
| layer | | A5 | — | — | 7 | — | — | — | — |
| | | A6 | — | — | — | 7 | — | — | — |
| | | A7 | — | — | — | — | 7 | — | — |
| | | A8 | — | — | — | — | — | 7 | — |
| | | A9 | — | — | — | — | — | — | 7 |
| | Stabilizer | B | — | — | — | 0.1 | — | — | — |
| | Cationic | C1 | 25 | 25 | 25 | 25 | 25 | 25 | 25 |
| | polymerizable | C2 | 25 | 25 | 25 | 25 | 25 | 25 | 25 |
| | compound | | | | | | | | |
| | Thermoplastic resin | D | 30 | 30 | 30 | 30 | 30 | 30 | 30 |
| | Coupling agent | E | 3 | 3 | 3 | 3 | 3 | 3 | 3 |
| | Filler | F1 | 5 | 5 | 5 | 5 | 5 | 5 | 5 |
| | | F2 | 15 | 15 | 15 | 15 | 15 | 15 | 15 |
| | Conductive particles | G | 45 | 45 | 45 | 15 | 45 | 45 | 45 |
| Second | Curing agent | A3 | 7 | — | — | — | — | — | — |
| adhesive | | A4 | — | 7 | — | — | — | — | — |
| layer | | A5 | — | — | 7 | — | — | — | — |
| | | A6 | — | — | — | 7 | — | — | — |
| | | A7 | — | — | — | — | 7 | — | — |
| | | A8 | — | — | — | — | — | 7 | — |
| | | A9 | — | — | — | — | — | — | 7 |
| | Stabilizer | B | — | — | — | 0.1 | — | — | — |
| | Cationic | C1 | 15 | 15 | 15 | 15 | 15 | 15 | 15 |
| | polymerizable | C3 | 15 | 15 | 15 | 15 | 15 | 15 | 15 |
| | compound | C4 | 10 | 10 | 10 | 10 | 10 | 10 | 10 |
| | | C5 | 30 | 30 | 30 | 30 | 30 | 30 | 30 |
| | Thermoplastic resin | D | 10 | 10 | 10 | 10 | 10 | 10 | 10 |
| | Coupling agent | E | 3 | 3 | 3 | 3 | 3 | 3 | 3 |
| | Filler | G2 | 20 | 20 | 20 | 20 | 20 | 20 | 20 |

TABLE 4

| | | | | | Compar- | Compar- | Compar- | Compar- | Compar- | Compar- | Compar- |
|---|---|---|---|---|---|---|---|---|---|---|---|
| | | Example | Example | Example | ative | ative | ative | ative | ative | ative | ative |
| | | 1 | 2 | 3 | Example 1 | Example 2 | Example 3 | Example 4 | Example 5 | Example 6 | Example 7 |
| Mounting temperature (° C.) | | 120 | 120 | 120 | 135 | 135 | 125 | 135 | 135 | 135 | 135 |
| Connection | Initial stage | A | B | B | D | E | E | E | E | E | D |
| resistance | After high- | B | B | C | E | E | E | E | E | E | E |
| | temperature | | | | | | | | | | |
| | and high- | | | | | | | | | | |
| | humidity test | | | | | | | | | | |

TABLE 5

| | | Example | Example | Example | Example | Example | Example |
|---|---|---|---|---|---|---|---|
| | | 4 | 5 | 6 | 7 | 8 | 9 |
| Mounting temperature (° C.) | | 120 | 120 | 120 | 120 | 120 | 120 |
| Connection | Initial stage | B | A | B | A | A | B |
| resistance | After high- | B | B | B | B | B | C |
| | temperature | | | | | | |
| | and high- | | | | | | |
| | humidity test | | | | | | |

TABLE 6

| | | | | | | Compar- | Compar- | Compar- | Compar- | Compar- |
|---|---|---|---|---|---|---|---|---|---|---|
| | | | Example | Example | Example | ative | ative | ative | ative | ative |
| | | | 1 | 2 | 3 | Example 1 | Example 2 | Example 3 | Example 4 | Example 5 |
| Mounting | Connection | Initial stage | A | A | B | E | E | E | E | E |
| temperature | resistance | After high- | A | A | B | — | — | — | — | — |
| 120° C. | | temperature and | | | | | | | | |
| | | high-humidity test | | | | | | | | |
| | | After high-speed | A | A | B | E | E | E | E | E |
| | | degradation test | | | | | | | | |
| | Appearance | Initial stage | A | A | A | C | C | C | C | C |
| | | After high- | B | A | A | — | — | — | — | — |
| | | temperature and | | | | | | | | |
| | | high-humidity test | | | | | | | | |
| | | After high-speed | B | B | A | C | C | C | C | C |
| | | degradation test | | | | | | | | |
| | Bonding | Adhesion | A | A | A | A | A | A | A | A |
| | property | Peelability | A | A | A | A | A | A | A | A |
| Mounting | Connection | Initial stage | A | A | A | A | B | E | E | E |
| temperature | resistance | After high- | A | A | A | B | B | E | E | E |
| 135° C. | | temperature and | | | | | | | | |
| | | high-humidity test | | | | | | | | |
| | | After high-speed | A | A | A | A | A | E | E | E |
| | | degradation test | | | | | | | | |
| | Appearance | Initial stage | A | A | A | A | A | C | C | C |
| | | After high- | B | A | B | B | A | C | C | C |
| | | temperature and | | | | | | | | |
| | | high-humidity test | | | | | | | | |
| | | After high-speed | C | B | B | A | A | C | C | C |
| | | degradation test | | | | | | | | |
| | Bonding | Adhesion | A | A | A | A | A | A | A | A |
| | property | Peelability | A | A | A | A | A | A | A | A |

TABLE 7

| | | | Example | Example | Example | Comparative | Comparative | Comparative | Comparative | Comparative |
|---|---|---|---|---|---|---|---|---|---|---|
| | | | 1 | 2 | 3 | Example 1 | Example 2 | Example 3 | Example 4 | Example 5 |
| Storage | Connection | Initial stage | A | A | B | A | B | E | E | E |
| stability | resistance | | | | | | | | | |
| | Appearance | Initial stage | A | A | B | A | B | B | B | B |
| | Bonding | Adhesion | C | C | B | A | A | B | B | A |
| | property | Peelability | B | A | A | A | A | A | A | A |

As shown in Tables 4 and 5, in the case of a curing agent containing a pyridinium salt, in which the pyridinium salt has a benzyl group at a 1-position and has an electron-withdrawing group at a 2-position and the benzyl group has an electron-donating group, an adhesive composition using this curing agent can be cured at a lower temperature (for example, 120° C.). Furthermore, an adhesive film using this adhesive composition can achieve excellent connection resistance in both the initial stage and the stage after the high-temperature and high-humidity test.

As shown in Table 6, in the case of a curing agent containing a pyridinium salt, in which the pyridinium salt has a benzyl group at a 1-position and has an electron-withdrawing group at a 2-position and the benzyl group has an electron-donating group, an adhesive film using this curing agent can achieve excellent connection resistance and excellent appearance in all of the initial stage, the stage after the high-temperature and high-humidity test, and the stage after the high-speed degradation test. Furthermore, an adhesive film using this curing agent can achieve, for example, both of excellent adhesion to a circuit member and excellent peelability of a substrate from the adhesive film. Further, as shown in Table 7, when this curing agent contains a pyridinium salt in which the number of electron-donating groups of the benzyl group disposed at the 1-position of the pyridinium salt is 3 and these electron-donating groups are all an alkyl group (when the curing agent A2 is used), as compared to the curing agent A1, storage stability is excellent, and excellent adhesion to a circuit member and excellent peelability of a substrate from the adhesive film can be maintained even after the adhesive film is stored at 40° C. for 15 hours. Note that, in Comparative Examples 1 and 2, the adhesive composition could be cured at a mounting temperature of 135° C. and storage stability was also excellent, but low-temperature curing at 120° C. could not be realized.

### REFERENCE SIGNS LIST

1: adhesive film for circuit connection, 1A: first adhesive layer, 1B: second adhesive layer, 2, 2A, 2B: adhesive component, 3, 3A: conductive particle, 4: first circuit member, 5: second circuit member, 6: connection portion, 7: insulating material, 10: structure, 41: first circuit board, 42: first electrode, 51: second circuit board, 52: second electrode.

1. A curing agent comprising a pyridinium salt,
wherein the pyridinium salt has a benzyl group at a 1-position and has an electron-withdrawing group at a 2-position, and
the benzyl group has an electron-donating group.

2. The curing agent according to claim 1, wherein the electron-withdrawing group is a cyano group or a halogeno group.

3. The curing agent according to claim 1, wherein the electron-donating group is an alkyl group or an alkoxy group.

4. The curing agent according to claim 1,
wherein the number of electron-donating groups of the benzyl group is 3, and
the electron-donating group is an alkyl group.

5. The curing agent according to claim 1,
wherein the pyridinium salt comprises a pyridinium cation and an anion, and
the anion is B(C₆F₅)₄⁻.

6. An adhesive composition comprising a pyridinium salt and a cationic polymerizable compound,
wherein the pyridinium salt has a benzyl group at a 1-position and has an electron-withdrawing group at a 2-position, and
the benzyl group has an electron-donating group.

7. The adhesive composition according to claim 6, wherein the electron-withdrawing group is a cyano group or a halogeno group.

8. The adhesive composition according to claim 6, wherein the electron-donating group is an alkyl group or an alkoxy group.

9. The adhesive composition according to claim 6,
wherein the number of electron-donating groups of the benzyl group is 3, and
the electron-donating group is an alkyl group.

10. The adhesive composition according to claim 6,
wherein the pyridinium salt comprises a pyridinium cation and an anion, and
the anion is B(C₆F₅)₄⁻.

11. The adhesive composition according to claim 6, wherein the cationic polymerizable compound comprises an epoxy compound.

12. The adhesive composition according to claim 6, wherein a content of the pyridinium salt is 0.1 to 40 parts by mass based on 100 parts by mass of the cationic polymerizable compound.

13. The adhesive composition according to claim 6, further comprising conductive particles.

14. An adhesive film for circuit connection, comprising an adhesive layer formed from the adhesive composition according to claim 6.

15. The adhesive film for circuit connection according to claim 14, wherein a content of the pyridinium salt is 1 to 20% by mass based on a total mass of the adhesive film for circuit connection.

16. An adhesive film for circuit connection, comprising a first adhesive layer and a second adhesive layer laminated on the first adhesive layer,
wherein at least one of the first adhesive layer and the second adhesive layer is a layer formed from the adhesive composition according to claim 6.

17. A connected structure comprising:
a first circuit member having a first electrode;
a second circuit member having a second electrode; and
a connection portion disposed between the first circuit member and the second circuit member and electrically connecting the first electrode and the second electrode to each other,
wherein the connection portion comprises a cured product of the adhesive film for circuit connection according to claim 14.

18. A method for producing a connected structure, the method comprising a step of interposing the adhesive film for circuit connection according to claim 14 between a first circuit member having a first electrode and a second circuit member having a second electrode, and thermocompression bonding the first circuit member and the second circuit member to electrically connect the first electrode and the second electrode to each other.
